(19) United States
(12) Patent Application Publication
(10) Pub. No.: US 2023/0154561 A1
(43) Pub. Date: May 18, 2023

(54) DEEP LEARNING SYSTEMS AND METHODS FOR PREDICTING STRUCTURAL ASPECTS OF PROTEIN-RELATED COMPLEXES

(71) Applicant: The Curators of the University of Missouri, Columbia, MO (US)

(72) Inventors: Jianlin Cheng, Columbia, MO (US); Elham Soltanikazemi, Columbia, MO (US); Raj Shekhor Roy, Columbia, MO (US); Farhan Quadir, Columbia, MO (US)

(21) Appl. No.: 17/988,461

(22) Filed: Nov. 16, 2022

Related U.S. Application Data
(60) Provisional application No. 63/280,037, filed on Nov. 16, 2021.

(51) Int. Cl. G16B 15/30; G16B 40/00; G06N 3/08; G16B 15/20; G06N 3/045

(52) U.S. Cl. CPC G16B 15/30; G16B 40/00; G06N 3/08; G16B 15/20; G06N 3/045

(57) ABSTRACT

Deep learning systems and methods for predicting structural aspects of protein-related complexes are described herein. An example method for predicting inter-chain distances of protein-related complexes includes receiving data associated with a protein-related complex, where the data associated with the protein-related complex includes at least one of a tertiary structural feature and a multiple sequence alignment (MSA)-derived feature. The method also includes inputting the data associated with the protein-related complex into a deep learning model. The method further includes predicting, using the deep learning model, an inter-chain distance map for the protein-related complex.

### CROSS-REFERENCE TO RELATED APPLICATIONS

This application claims the benefit of U.S. provisional patent application No. 63/280,037, filed on Nov. 16, 2021, and titled “Deep Learning Systems and Methods for Predicting Structural Aspects of Protein-Related Complexes,” the disclosure of which is expressly incorporated herein by reference in its entirety.

### STATEMENT REGARDING FEDERALLY FUNDED RESEARCH

This invention was made with government support under DE-AR0001213 awarded by the U.S. Department of Energy, GM093123 awarded by the National Institutes of Health, and 1759934 awarded by the National Science Foundation. The government has certain rights in the invention.

### BACKGROUND

Proteins are essential molecules in biological systems. They interact to form protein complexes to carry out various biological functions such as catalyzing chemical reactions and transducting signals. Knowledge of a protein's structure provides insight into how it can interact with other proteins, DNA, RNA, and other small molecules (e.g., ligands).

These structures have been a focus of computational biology. For example, since “wet” laboratory experiments with actual proteins are time-consuming and expensive, accurate computational tools are critical for being able to analyze protein structures. Genomics projects and other research have contributed large amounts of publicly accessible biological data including various protein-related data (e.g., protein sequence data). Researchers around the world contribute and have access to such data pools. This data includes, for example, 3D structure data of proteins and other molecules.

The methods and systems described herein can be applied to study, engineer and design proteins and protein-related complexes to improve and modulate protein function in many domains including but not limited to medicine, drug design, protein design, agriculture, bioenergy production, and biotechnology development.

### SUMMARY

An example computer-implemented method for predicting inter-chain distances of protein-related complexes is described herein. The method includes receiving data associated with a protein-related complex, where the data associated with the protein-related complex includes at least one of a tertiary structural feature and a multiple sequence alignment (MSA)-derived feature. The method also includes inputting the data associated with the protein-related complex into a deep learning model. The method further includes predicting, using the deep learning model, an inter-chain distance map for the protein-related complex.

In some implementations, the tertiary structural feature includes an intra-chain distance map for at least one monomer of the protein-related complex.

In some implementations, the MSA-derived feature includes a plurality of residue-residue co-evolutionary scores for at least one monomer of the protein-related complex. Alternatively or additionally, the MSA-derived feature includes a position-specific scoring matrix (PSSM) for at least one monomer of the protein-related complex.

Optionally, in some implementations, the data associated with the protein-related complex includes the tertiary structural feature and the MSA-derived feature.

In some implementations, the protein-related complex is a homodimer, and the data associated with the protein-related complex includes information related to a single monomer.

In some implementations, the protein-related complex is a heterodimer, and the data associated with the protein-related complex includes respective information related to a plurality of monomers.

In some implementations, the protein-related complex includes a first protein and a second protein, where the first protein includes a first chain of residues and the second protein includes a second chain of residues. In these implementations, the inter-chain distance map includes a plurality of residue-to-residue distances between residues of the first and second proteins.

In some implementations, the protein-related complex includes a protein and a ligand. In these implementations, the inter-chain distance map includes a plurality of atom-to-atom distances between atoms of the protein and the ligand.

In some implementations, the inter-chain distance map is a heavy atom distance map.

In some implementations, the inter-chain distance map is a Ce distance map.

In some implementations, the inter-chain distance map for the protein-related complex is at least one matrix including a respective probability of a residue-residue distance between respective residues of a pair of monomers of the protein-related complex in each of a plurality of distance bins.

In some implementations, the deep learning model includes an artificial neural network (ANN), where the ANN includes a plurality of layers. The layers include at least two of a convolution layer, an attention layer, a dense layer, a dropout layer, a residual layer, a normalization layer, and an embedding layer. Optionally, the ANN is a transformer network, where the transformer network includes a plurality of 2D transformer blocks, the 2D transformer blocks including a plurality of attention generation layers, a plurality of attention convolution layers, and a plurality of projection layers.

In some implementations, the deep learning model includes an attention-powered residual neural network.

In some implementations, the deep learning model includes a dilated convolutional residual neural network.

In some implementations, the deep learning model includes a plurality of convolutional neural networks (CNNs).

In some implementations, the deep learning model includes a graph neural network (GNN).

In some implementations, the method further includes generating a three-dimensional (3D) structure for the protein-related complex using the inter-chain distance map as a restraint. Optionally, the 3D structure is generated using a gradient decent (GD) optimization. Alternatively, the 3D structure is generated using a simulated annealing algorithm, a Monte Carlo simulation, or deep reinforcement learning (DRL). In some implementations, the 3D structure is optionally a quaternary structure. For example, the protein-related complex includes a first protein and a second protein, where the first protein includes a first chain of residues, the second protein includes a second chain of residues, and the inter-chain distance map for the protein-related complex includes a plurality of residue-to-residue distances between residues of the first and second proteins. Alternatively, in some implementations, the protein-related complex includes a protein and a ligand, and the inter-chain distance map includes a plurality of atom-to-atom distances between atoms of the protein and the ligand.

In some implementations, the method further includes training the deep learning model using a dataset, where the dataset includes known inter-chain parameters for a plurality of known protein-related complexes.

In some implementations, each known protein-related complex includes a first protein and a second protein, where the first protein includes a first chain of residues, the second protein includes a second chain of residues, and the inter-chain parameters include a plurality of residue-to-residue distances between residues of the first and second proteins.

Alternatively or additionally, in some implementations, each known protein-related complex includes a first protein and a second protein, where the first protein includes a first chain of residues, the second protein includes a second chain of residues, and the inter-chain parameters include a plurality of residue-to-residue contacts between residues of the first and second proteins.

Alternatively or additionally, in some implementations, the protein-related complex includes a protein and a ligand, and the inter-chain parameters includes a plurality of atom-to-atom distances between atoms of the protein and the ligand.

Alternatively or additionally, in some implementations, the protein-related complex includes a protein and a ligand, and the inter-chain parameters includes a plurality of atom-to-atom contacts between atoms of the protein and the ligand.

An example system for predicting inter-chain distances of protein-related complexes is described herein. The system includes a deep learning model. The system also includes a processor and a memory, where the memory has computer-executable instructions stored thereon. The processor is configured to input data associated with a protein-related complex into the deep learning model, where the data associated with the protein-related complex includes at least one of a tertiary structural feature and a multiple sequence alignment (MSA)-derived feature. The processor is further configured to receive, from the deep learning model, an inter-chain distance map for the protein-related complex. The inter-chain distance map for the protein-related complex is predicted by the deep learning model.

In some implementations, the processor is a plurality of processors. Optionally, the processors are part of a distributed computing architecture. Alternatively or additionally, the memory is a plurality of memories.

An example computer-implemented method for predicting a three-dimensional (3D) structure of protein-related complexes is described herein. The method includes receiving first data associated with a protein-related complex, and receiving second data including a plurality of inter-chain parameters for the protein-related complex. The method also includes performing structure optimization on the first data using the second data as a restraint. The method further includes predicting the 3D structure of the protein-related complex based on the structure optimization, where the structure optimization includes gradient descent (GD) optimization.

In some implementations, the protein-related complex includes a first protein and a second protein, where the first protein includes a first chain of residues and the second protein includes a second chain of residues. Optionally, the second data includes a plurality of residue-to-residue distances between residues of the first and second proteins. Alternatively, the second data optionally includes a plurality of residue-to-residue contacts between residues of the first and second proteins.

In some implementations, the protein-related complex includes a protein and a ligand. Optionally, the second data includes a plurality of atom-to-atom distances between atoms of the protein and the ligand. Alternatively, the second data optionally includes a plurality of atom-to-atom contacts between atoms of the protein and the ligand.

An example system for predicting a three-dimensional (3D) structure of protein-related complexes is described herein. The system includes a processor and a memory, where the memory has computer-executable instructions stored thereon. The processor is configured to receive first data associated with a protein-related complex, and receive second data including a plurality of inter-chain parameters for the protein-related complex. The processor is also configured to perform structure optimization on the first data using the second data as a restraint, and predict the 3D structure of the protein-related complex based on the structure optimization.

In an example implementation, an artificial neural network such as a deep-learning neural network is trained with training data that comprises known protein-related complexes with known inter-chain parameters to generate a model that predicts those inter-chain parameters for any protein-related complexes.

As an example, the known protein-related complexes can be known protein complexes. Each protein complex can comprise a first protein and a second protein (e.g., a protein dimer), where the first protein comprises a first chain of residues and where the second protein comprises a second chain of residues. If the two chains are the same, the protein complex is a homodimer; otherwise, it is a heterodimer. It is worth noting that a protein complex comprised of more than two protein chains can be treated as multiple pairs of chains (dimers). As an example, the known protein complexes used by the neural network to train the model can be protein homodimers. In an example implementation where the known protein-related complexes are known protein complexes, the known and predicted inter-chain parameters can comprise residue-to-residue distances between the residues of the first and second proteins. In another example implementation where the known protein-related complexes are known protein complexes, the known and predicted inter-chain parameters can comprise residue-to-residue contacts (i.e., binary representation, such as 1 if pair of residues separated by less than threshold (e.g., 6 Å or 8 Å) or 0 otherwise) between the residues of the first and second proteins.

As another example, the known protein-related complexes can be known protein-ligand complexes. Each protein complex can comprise a protein and a ligand. The ligand can take the form of a small molecule (e.g., a drug), RNA, DNA, or a peptide. In an example implementation where the known protein-related complexes are known protein-ligand complexes, the known and predicted inter-chain parameters can comprise atom-to-atom distances between the atoms of the protein and the ligand. In another example implementation where the known protein-related complexes are known protein-ligand complexes, the known and predicted inter-chain parameters can comprise atom-to atom contacts (i.e., binary representations, such as 1 if pair of residues separated by less than threshold (e.g., 6 Å or 8 Å) or 0 otherwise) between the atoms of the protein and the ligand.

In another example implementation, techniques where the trained model that is predictive of inter-chain parameters for protein-related complexes is used to predict the inter-chain parameters of a protein-related complex, preferably where such protein-related complex was not used in training, are disclosed. As noted above, as an example, the protein-related complex can be a protein complex, and the predicted inter-chain parameters can be (1) predicted residue-to-residue distances between residues of the different proteins of the protein complex and/or (2) predicted residue-to-residue contacts (i.e., binary representations, such as 1 if pair of residues separated by less than threshold (e.g., 6 Å or 8 Å) or 0 otherwise) between residues of the different proteins of the protein complex. Furthermore, as noted above, as an example, the protein-related complex can be a protein-ligand complex, and the predicted inter-chain parameters can be (1) predicted atom-to-atom distances between atoms of the protein and ligand of the protein-ligand complex and/or (2) predicted atom-to-atom contacts (i.e., binary representations, such as 1 if pair of residues separated by less than threshold (e.g., 6 Å or 8 Å) or 0 otherwise) between atoms of the protein and ligand of the protein-ligand complex.

In another example implementation, techniques where structural optimization such as deep reinforcement learning or gradient descent optimization is performed on a protein complex using inter-chain distances or inter-chain contacts as restraints for the structural optimization to predict a quaternary structure for the protein complex are disclosed. The inter-chain distances/contacts that are used as the restraints can be predicted inter-chain distances/contacts such as those predicted using the neural network-based trained model as discussed above.

In yet another example implementation, techniques where structural optimization such as deep reinforcement learning or gradient descent optimization is performed on a protein-ligand complex using inter-atom distances or inter-atom contacts as restraints for the structural optimization to predict a 3D structure for the protein-ligand complex are disclosed. As noted above, the inter-atom distances/contacts that are used as the restraints can be predicted inter-atom distances/contacts such as those predicted using the neural network-based trained model as discussed above.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram illustrating a deep learning model operating in inference mode according to an implementation described herein.

FIG. 2 is a flowchart illustrating example operations for predicting inter-chain distances of protein-related complexes according to an implementation described herein.

FIG. 3 is a flowchart illustrating example operations for predicting a three-dimensional (3D) structure of protein-related complexes according to an implementation described herein.

FIG. 4 is an example computing device.

FIG. 5 is a histogram of the precision of the top L/10 contact predictions for the heterodimers in the HeteroTest2 dataset described in Example 1. The X-axis is the four precision intervals from 0% to 100%. The Y-axis is the number of heterodimers whose contact precision falls in each interval. Each interval has 40, 2, 1, and 12 heterodimers, respectively.

FIG. 6 is a comparison between CDPred_PLM (blue, left bar) and CDPred_ESM (orange, right bar) on four different test datasets described in Example 1. The y-axis is the top L/10 contact prediction precision, and the x-axis is the four different test datasets.

FIG. 7 is a comparison of using AlphaFold predicted tertiary structure (blue, “PRED”) and true tertiary structure (yellow, “TRUE”) to generate intra-chain distance maps as input for predicting inter-chain distance maps on the four datasets described in Example 1. Top L/2 contact prediction precision on the datasets is reported.

FIG. 8 is the plot of the precision of top L/5 inter-chain contact predictions against their average contact probability predicted by CDPred for each target in the four test datasets (HomoTest1, HomoTest2, HeteroTest1, HeteroTest2) described in Example 1.

FIGS. 9A-9D illustrate the prediction for homodimer T0991 with a shallow MSA as described in Example 1. FIG. 9A is the intra-chain distance map of the monomer predicted by AlphaFold.

FIG. 9B is the true intra-chain distance map of the monomer. FIG. 9C is the inter-chain contact map predicted by CDPred. FIG. 9D is the true inter-chain contact map.

FIG. 10 illustrates the CDPred architecture as described in Example 1. CDPred simultaneously uses the tertiary structural information (i.e., intra-chain distance map of monomers), sequential information (PSSM), and residue-residue co-evolutionary information (i.e., co-evolutionary scores calculated by CCMpred and attention maps by MSA transformer) as input to predict inter-chain distance maps. The dimension of the input for homomer dimer is L*L*186 (L is the length of the monomer sequence), while the dimension of the input for heterodimer is (L1+L2)×(L1+L2)*186 (L1 and L2 are the length of the two different monomers in the heterodimer). Each of the two output matrices has the same dimension as the input except for the number of output channels. The number of the output channels of the output layer is 42, storing the predicted probability of the distance in 42 distance bins. Two output matrices are generated, representing the two kinds of predicted inter-chain distance maps.

FIG. 11 is Table 1 described in Example 1, which shows the precision of top 5, top 10, top L/10, top L/5, and top L contact predictions, accuracy order (AccOrder), accuracy rate (AccRate), and AUC (area under receiver operating characteristic curve) score on the HomoTest1 test dataset for DNCON2_inter, DeepComplex, DeepHomo, GLINTER, and three versions of CDPred. L: sequence length of a monomer in a homodimer. Bold numbers denote the best results. AccOrder is the rank of the first correct contact prediction divided by the number of residues of a dimer. The smaller is AccOrder, the better is the performance. AccRate is the percentage of dimers for which at least one of the top 10 inter-chain contact predictions is correct.

FIG. 12 is Table 2 described in Example 1, which shows the precision of top 5, top 10, top L/10, top L/5, and top L contact predictions, accuracy order, accuracy rate, and AUC score on the HomoTest2 test dataset for DeepHomo, GLINTER, and CDPred predictors. Bold numbers denote the highest precision.

FIG. 13 is Table 3 described in Example 1, which shows the evaluation of contact predictions on the HeteroTest1 test dataset for the DeepComplex, GLINTER and CDPred. Ls: the sequence length of the shorter monomer in a heterodimer. Bold numbers denote the best result.

FIG. 14 is Table 4 described in Example 1, which shows the evaluation of contact predictions on the HeteroTest2 test dataset for the DeepComplex, GLINTER, and CDPred. Ls: the sequence length of the shorter monomer in a heterodimer. Bold numbers denote the best result.

FIG. 15 illustrates the DRCon deep learning architecture for interchain contact prediction as described in Example 2.

FIGS. 16A-16B illustrates the DNCON2 deep learning architecture for interchain contact prediction as described in Example 3. FIG. 16A illustrates a total of six CNNs are used, i.e. five in the first level to predict preliminary contact probabilities at 6, 7.5, 8, 8.5 and 10 Å distance thresholds separately by using an input volume of a protein as input, and one in the second level that take both the input volume and the 2D contact probabilities predicted in the first level to make final predictions. FIG. 16B illustrates each of the six CNN networks have the same architecture, which has six hidden convolutional layers and one output layer consisting of 16 filters of 5 by 5 size and one output layer.

FIG. 17 is a block diagram illustrating the DeepInteract overview as described in Example 4. The DeepInteract pipeline separates interface contact prediction into two tasks: (1) learning new node representations h_{A} and h_{B} for pairs of residue protein graphs and (2) convolving over h_{A} and h_{B} interleaved together to predict pairwise contact probabilities.

FIG. 18 is a block diagram illustrating the Geometric Transformer overview as described in Example 4. Notably, the final layer of the Geometric Transformer removes the edge update path since, in this formulation of interface prediction, only graph pairs' node representations h_{A} and h_{B} are directly used for the final interface contact prediction.

FIG. 19 is a block diagram illustrating the Conformation module overview as described in Example 4. The Geometric Transformer uses a conformation module in each layer to evolve proteins graphs' geometric representations via repeated gating and a final series of residual connection blocks.

FIG. 20 illustrates an example approach for deep reinforcement learning (DRL) to predict a 3D structure (quaternary structure) of a protein complex according to an implementation described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

The term “protein-related complex” refers to: (i) two or more interacting proteins, such as a first protein including a first chain of residues and a second protein includes a second chain of residues; or (ii) two or more interacting proteins and ligands, such as a protein and a ligand. It should be understood that a protein-related complex has a three-dimensional (3D) structure.

The term “residue” refers to a single unit that makes up a polymer, such as an amino acid in a polypeptide chain or protein (e.g., a residue is an individual organic compound (e.g., amino acid) that comprises some of the building blocks of complete proteins).

The term “residue-residue contact” (sometimes referred herein to as “contact”) refers to a pair of spatially close residues in a protein-related complex.

The term “intra-chain contact” refers to a residue-residue contact within a single protein chain, while the term “inter-chain contact” refers to a residue-residue contact between two protein chains.

The term “intra-chain distance” refers to a distance between a pair of residues within a single protein chain, while the term “inter-chain distance” refers to a distance between a pair of residues between two protein chains.

The term “distance map” refers to a representation (e.g., a two-dimensional (2D) tensor, which is sometimes referred to as a “matrix”) of the distances between all possible residue pairs of a protein-related complex. Distance maps include intra-chain distance maps and inter-chain distance maps.

The term “contact map” refers to a binary representation (e.g., a two-dimensional (2D) tensor, which is sometimes referred to as a “matrix”) of the distances between all possible residue pairs of a protein-related complex. For residue i and j, the ij element of the contact map may be set to 1 if the residues are closer than a threshold (e.g., between 6-12 angstrom (Å), optionally 6 Å or 8 Å) and otherwise may be set to 0. Contact maps include intra-chain contact maps and inter-chain contact maps.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, deep artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

### INTRODUCTION

For protein complexes comprising two or more interacting proteins, residue-residue contacts (or simply “contacts”) in protein 3D structures are pairs of spatially close residues. A residue refers to a single unit that makes up a polymer, such as an amino acid in a polypeptide chain or protein (e.g., a residue is an individual organic compound (e.g., amino acid) that comprises some of the building blocks of complete proteins). A 3D structure of a protein can be expressed as x-, y-, and z-coordinates of the amino acids' atoms, and as such contacts can be defined in terms of distance(s). Some contacts may be inter-chain (between two protein chains), while other contacts may be intra-chain (within a protein chain). Other factors to consider include the total number of contacts in a protein and the coverage of contacts (e.g., distribution of contacts over the structure of a protein).

A protein distance map can be generated to represent the distances between all possible (e.g., amino acid) residue pairs of a 3D protein structure. Alternatively or additionally, a protein contact map can be generated to represent the distances between all possible (e.g., amino acid) residue pairs of a 3D protein structure. A protein distance map and a protein contact map, which is a binary representation, provide a simplified representation of a protein structure compared to full atomic coordinates, which in turn makes them more amenable to prediction via machine learning techniques, and hence useful in reconstructing coordinates of a protein using its contact map.

Recently, artificial intelligence (“AI”) in the form of machine learning (“ML”) has been used to facilitate computational modeling of protein structures, and has contributed to an increase in the precision of contact prediction. Such predicted contacts can be used to build 3D models (e.g., from scratch (aka not from a template)). For example, predicted protein reside-residue information can be used to build 3D models and predict protein folds. Some machine learning techniques can predict inter-chain distances, but they are not trained for inter-chain contacts, rather they are trained on intra-chain contacts (same chain contacts). For example, intra-chain contacts and distances are useful in tertiary structure modeling for a single protein, but cannot be used to build quaternary structures of protein complexes comprised of multiple proteins. While predicted intra-chain residue-residue contacts have been successfully applied to predict inter-chain contacts between a small portion of proteins, these methods (e.g., direct coupling analysis, “DCA”) require deep multiple sequence alignments (MSAs) of a pair of interacting proteins (e.g., dimers) as input, which are difficult to obtain because there are not many known protein complexes available to generate MSAs of sufficient depth for a pair of proteins. Obtaining MSAs of interlogs of sufficient depth is difficult because there are fewer known protein complexes than known monomers. Moreover, prediction accuracy is lacking in these conventional techniques, especially as to predicting quaternary structures of protein complexes comprising of multiple chains, due to lack of advanced deep learning techniques relating to quaternary structure applications. Techniques that are not specially designed for quaternary structure prediction and that are not trained on protein complex data fail to achieve the necessary accuracy to be generally useful.

In an effort to improve upon these shortcomings in the art, described herein are various techniques that use machine learning to improve how inter-chain parameters (e.g., inter-chain contacts/distances between residues) of protein-related complexes are predicted.

Predicting Inter-Chain Distances of Protein-Related Complexes

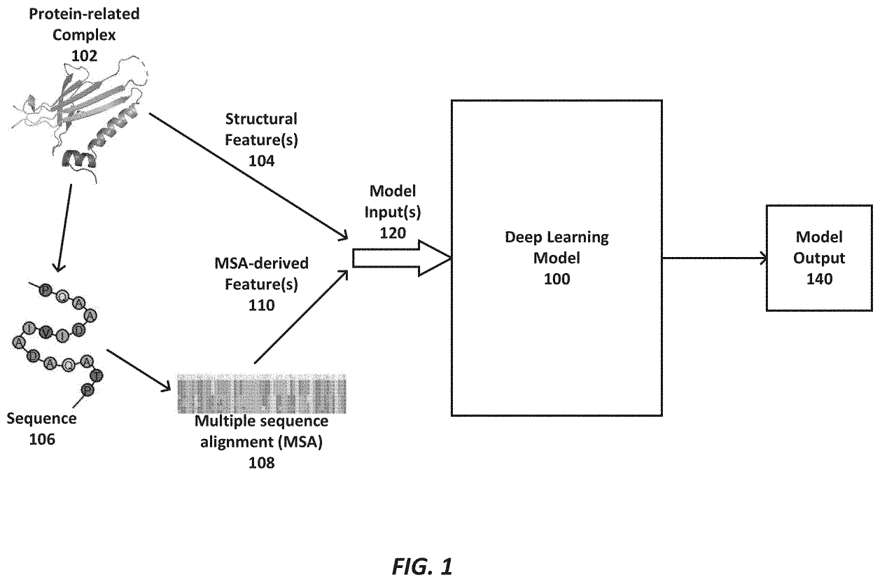

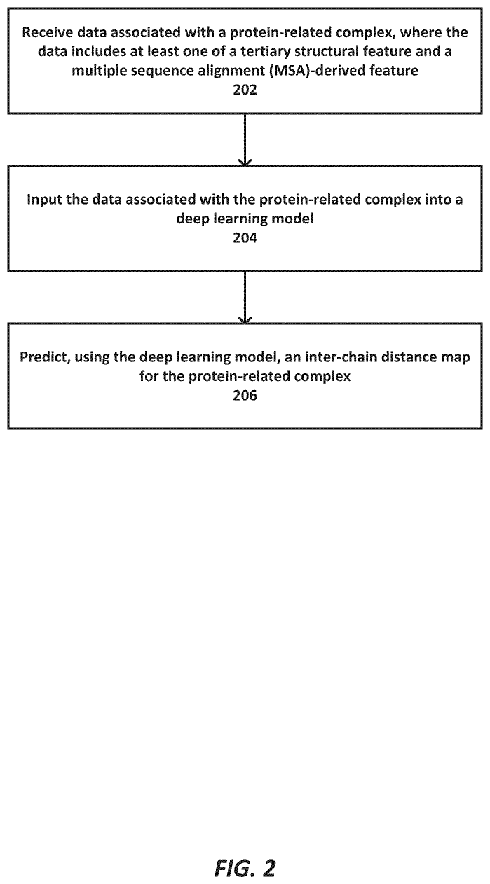

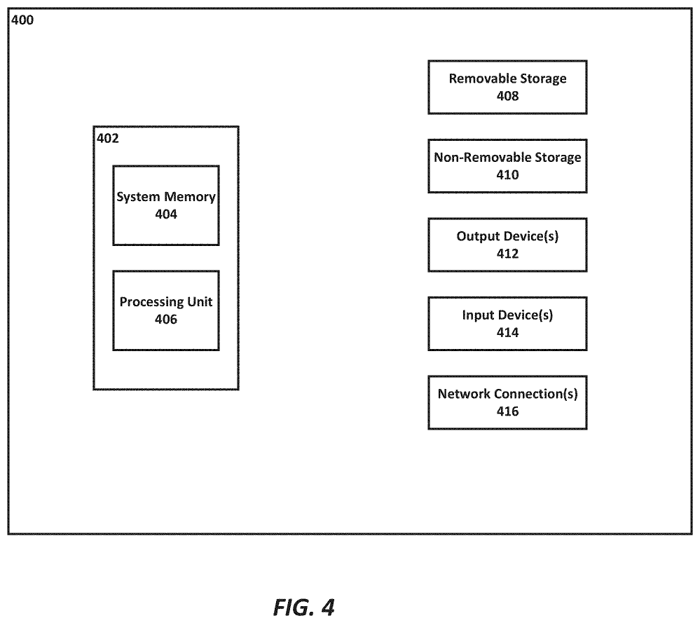

Deep learning systems and methods for predicting inter-chain distances of protein-related complexes are described herein. Referring now to FIGS. 1 and 2, a method for predicting inter-chain distances of protein-related complexes is shown. The predicted inter-chain distances can be applied to study, engineer and design proteins and protein-related complexes to improve and modulate protein function in many domains including but not limited to medicine, drug design, protein design, agriculture, bioenergy production, and biotechnology development. This disclosure contemplates that the logical operations can be performed using at least one processor and memory (see e.g., computing device 400 shown in FIG. 4). Optionally, the processor is a plurality of processors. Optionally, the processors are part of a distributed computing architecture. Alternatively or additionally, the memory is optionally a plurality of memories. The deep learning systems and methods for predicting inter-chain distances of protein-related complexes are different than, and also offer improvements over, existing deep learning methods. For example, the deep learning systems and methods described herein are configured to predict inter-chain (i.e., between two protein chains) distances rather than intra-chain (i.e., within a protein chain) distances as predicted by existing deep learning methods such as AlphaFold. Additionally, the deep learning systems and methods described herein are configured to predict inter-chain distances (e.g., an inter-chain distance map) rather than binary inter-chain contacts (contact or no contact) (e.g., inter-chain contact map) as predicted by existing deep learning methods such as DeepHomo and GLINTER. For example, as described in Example 1, the deep learning systems and methods described in Example 1 have been rigorously tested on two homodimer test datasets and two heterodimer test datasets. For these datasets, the deep learning systems and methods described in Example 1 yield much higher accuracy than DeepHomo and GLINTER. The deep learning systems and methods described herein therefore in Example 1 have advantages over existing deep learning methods such as AlphaFold, which is merely configured to predict intra-chain parameters (contacts or distances), or DeepHomo and GLINTER, which are merely configured to predict inter-chain contacts.

Referring now to FIG. 1, a block diagram illustrating a deep learning model 100 is shown. In FIG. 1, the deep learning model 100 is operating in inference mode. The deep learning model 100 has therefore been trained with a data set (or “dataset”). The deep learning model 100 is a supervised learning model. Supervised learning models are known in the art. A supervised learning model “learns” a function that maps a model input 120 (also known as feature or features) to a model output 140 (also known as target or targets) during training with a labeled data set. Deep learning model training is discussed in further detail below.

The deep learning model 100 is trained to map the model input 120 to the model output 140. In examples described herein, the model input 120 is data associated with a protein-related complex, and the model output 140 is an inter-chain distance map for the protein-related complex. The data associated with the protein-related complex includes one or more “features” that are input into the deep learning model 100, which predicts the inter-chain distance map for the protein-related complex. The inter-chain distance map for the protein-related complex is therefore the “target” of the machine learning model 100.

Referring now to FIG. 2, at step 202, data associated with a protein-related complex is received, for example, by a computing device. An example protein-related complex 102 is illustrated in FIG. 1. As described herein, in some implementations, a protein-related complex is a protein complex including a first protein and a second protein, where the first protein includes a first chain of residues and the second protein includes a second chain of residues. Optionally, the protein-related complex is a homodimer (i.e., formed by two identical proteins). In this case, the data associated with the protein-related complex includes information related to a single monomer. Alternatively, the protein-related complex is a heterodimer (i.e., formed by two identical proteins). In this case, the data associated with the protein-related complex includes respective information related to a plurality of monomers. It should be understood that a protein-related complex including two proteins (i.e., homodimers and heterodimers) is provided only as an example. This disclosure contemplates that the protein-related complex may include more than two proteins. In other implementations, the protein-related complex is a protein-ligand complex including a protein and a ligand. It should be understood that a protein-related complex including a protein and a ligand is provided only as an example. This disclosure contemplates that the protein-related complex may include more than one protein and more than one ligand.

As described herein, the data associated with the protein-related complex includes one or more features, which serve as the model input 120 to the deep learning model 100. It should be understood that the one or more features are stored in a tensor, i.e., a container that houses data in “N” dimensions. In the implementations described herein, the one or more features are stored in multidimensional matrices, such as two-dimensional (2D) tensors with multiple channels. For example, the one or more features can be stored in an L×L×N tensor, where L is the length of the sequence of a monomer for a homodimer or the sum of the length of the sequences of two monomers (L=L₂+L₂) for a heterodimer, and N is the number of feature channels for each pair of residues. As described below, the 2D tensors with multiple channels (e.g., model input 120) can be fed into an input layer of the deep learning model 100. It should be understood that 2D tensors with multiple channels are provided only as an example of a multidimensional tensor for the model input 120. This disclosure contemplates that the model input 120 may be a tensor with a different number of dimensions.

The one or more features may include, but are not limited to: multiple sequence alignments (MSAs); residue-residue co-evolutionary features of proteins describing the level of the correlated mutations between any two residues of proteins during evolution; structural features (e.g., secondary structures and solvent accessibility of residues); coordinates of residues; residue conservation scores calculated from MSAs; intra-chain contacts; and/or intra-chain distances. This disclosure contemplates that features can be converted into numerical values describing the relationships between any two residues between two proteins in a dimer by feature extraction programs or deep learning feature embedding techniques. Such features can be stored in a tensor. It should be understood that the features described above are provided only as examples.

In some implementations, the data associated with the protein-related complex includes a structural feature associated with the protein-related complex, such a tertiary structural feature (see e.g., homodimers). Optionally, the data associated with the protein-related complex includes a plurality of structural features (see e.g., heterodimers). For example, this implementation is described in detail in Example 2 and Example 4. A tertiary structural feature may be an intra-chain distance map for a monomer. Alternatively, a tertiary structural feature may be an intra-chain contact map for a monomer. For homodimers, the tertiary structural feature associated with a single monomer is the feature. For heterodimers, a respective tertiary structural feature associated with each of the monomers are the features. An example protein-related complex 102 and structural feature 104 are illustrated in FIG. 1.

In some implementations, the data associated with the protein-related complex includes a multiple sequence alignment (MSA)-derived feature associated with the protein-related complex. For example, this implementation is described in detail in Example 3. MSAs can be generated from the sequences of the protein-related complex using MSA tools known in the art. For homodimers, this includes searching the sequence of a monomer against a database to generate the MSA. For heterodimers, this includes searching the sequences of each different monomer against a database to generate the respective MSA for each of the monomers and then pairing the respective MSAs to produce the MSA for the heterodimer. MSA generation is discussed in further detail in Example 1. An example protein-related complex 102, sequence 106, MSA 108, and MSA-derived feature 110 are illustrated in FIG. 1. Optionally, the MSA-derived feature includes a plurality of residue-residue co-evolutionary scores for at least one monomer of the protein-related complex. For homodimers, the MSA-derived feature includes co-evolutionary scores for a single monomer. For heterodimers, the MSA-derived features include respective co-evolutionary scores for each of the different monomers. Alternatively or additionally, the MSA-derived feature optionally includes a position-specific scoring matrix (PSSM) for at least one monomer of the protein-related complex. For homodimers, the MSA-derived feature includes a PSSM for a single monomer. For heterodimers, the MSA-derived features include respective PSSMs for each of the different monomers.

In some implementations, the data associated with the protein-related complex optionally includes both tertiary structural and MSA-derived features. For example, this implementation is described in detail in Example 1, where the data associated with the protein-related complex is stored an L×L×N tensor, where L is the length of the sequence of a monomer for a homodimer or the sum of the length of the sequences of two monomers (L=L₂+L₂) for a heterodimer, and N is the number of feature channels for each pair of residues. The features stored in such tensor include (1) the tertiary structure information of monomers in the form of intra-chain distance map, (2) pair-wise co-evolutionary features, and (3) sequential amino acid conservation features.

Referring again to FIG. 2, at step 204, the data associated with the protein-related complex is input into a deep learning model. An example deep learning model 100 is shown in FIG. 1. As described herein, the deep learning model can include an artificial neural network (ANN). An ANN is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

A convolutional neural network (CNN) is a type of deep neural network that has been applied, for example, to image analysis applications. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. A graph convolutional neural networks (GCNN) is CNNs that have been adapted to work on structured datasets such as graphs.

It should be understood that an artificial neural network (e.g., ANN, CNN, GCNN) is provided only as an example machine learning model. Optionally, the machine learning models described herein are deep learning models. Machine learning models are known in the art and are therefore not described in further detail herein.

Optionally, in some implementations, the deep learning model includes an attention-powered residual neural network, which is described in detail in Example 1. In this implementation, the deep learning model includes an ANN with a plurality of layers. The layers include at least two of a convolution layer, an attention layer, a dense layer, a dropout layer, a residual layer, a normalization layer, and an embedding layer. Optionally, the ANN is a transformer network, where the transformer network includes a plurality of 2D transformer blocks, the 2D transformer blocks including a plurality of attention generation layers, a plurality of attention convolution layers, and a plurality of projection layers. It should be understood that an attention-powered residual neural network is provided only as an example. This disclosure contemplates that the deep learning model may include other ANN configurations including, but not limited to, those described in further detail below.

Referring again to FIG. 2, at step 206, an inter-chain distance map for the protein-related complex is predicted using the deep learning model. As described herein, the deep learning model has previously been trained to map the model input (e.g., the data associated with the protein-related complex) to the model output (e.g., the inter-chain distance map for the protein-related complex). In other words, the deep learning model is operating in inference mode at step 206. In implementations where the protein-related complex includes a plurality of proteins (i.e., protein complex), the inter-chain distance map includes a plurality of residue-to-residue distances between residues of a first protein and a second protein. In implementations where the protein-related complex includes a protein and a ligand (i.e., protein-ligand complex), the inter-chain distance map includes a plurality of atom-to-atom distances between atoms of the protein and the ligand. As described herein, an inter-chain distance map is a representation of the distances between all possible residue pairs of the protein-related complex. Optionally, the inter-chain distance map is a heavy atom distance map. Alternatively, the inter-chain distance map is optionally a C_{b} distance map.

In some implementations, the inter-chain distance map for the protein-related complex can be at least one matrix (i.e., a 2D tensor). Optionally, the at least one matrix includes a respective probability of a residue-residue distance between respective residues of a pair of monomers of the protein-related complex in each of a plurality of distance bins. Each distance bin stores a respective probability of a residue-residue distance between respective residues of a pair of monomers of the protein-related complex. For example, the at least one matrix may include 42 distance bins, where 40 bins from 2 to 22 Å with a bin size of 0.5 Å, 1 bin for 0-2 Å, and 1 bin for >22 Å. This implementation is described in further detail in Example 1. It should be understood that the number of bins and bin sizes are provided only as examples. This disclosure contemplates using a matrix having a different number of bins and/or bins of different sizes.

Optionally, the inter-chain distance map for the protein-related complex as predicted by the deep learning model at step 206 can be converted into an inter-chain contact map. As described herein, an inter-chain contact map is a binary representation of the distances between all possible residue pairs of a protein-related complex. For example, an inter-chain distance map can be converted into an inter-chain contact map by considering any two residues whose distance is less than a threshold as a contact (value=1) and others as non-contacts (value=0). An inter-chain distance map distance map therefore may be presumed to contain more information than a binary inter-chain contact map. Different distance thresholds (e.g., 6 Angstroms or 8 Angstroms) may be used, depending on a users' need. For example, for residue i and j, the ij element of the inter-chain contact map may be set to 1 if the residues are closer than a threshold (e.g., between 6-12 angstrom (Å), optionally 6 Å or 8 Å) and otherwise may be set to 0.

Optionally, the inter-chain distance map for the protein-related complex as predicted by the deep learning model at step 206 can be used to generate a three-dimensional (3D) structure for the protein-related complex. In implementations with protein complexes, the 3D structure is optionally a quaternary structure. Optionally, the 3D structure is generated using a gradient decent (GD) optimization. This implementation is described in detail in Example 5. As described herein, the GD optimization is an iterative optimization process to adjust the positions of protein chains to satisfy the inter-chain contacts, where the inter-chain distance map for the protein-related complex as predicted by the deep learning model at step 206 is used as a restraint. It should be understood that GD optimization is provided only as an example technique for generating 3D structure for the protein-related complex. This disclosure contemplates using other techniques to generate 3D structure for the protein-related complex including, but not limited to, a simulated annealing algorithm, a Monte Carlo simulation, or deep reinforcement learning (DRL). For example, DRL uses a self-learning process to adjust the position of the protein chains. Once an action (e.g., rotation and translation of a protein chain) is taken, a reward is returned based on how well the action may change the structure of the protein-related complex. An action that makes the structure of the protein-related complex satisfy more inter-chain contacts immediately or in the long-term receives a positive reward; otherwise, it receives a negative reward. The rewards obtained in the process are used to train a deep neural network to predict the value of different actions. As the self-learning process proceeds, the deep neural network gets better and better and can predict the value of the actions better. Positive actions selected by the deep neural network can adjust the positions of protein chains better to reach a structure that is very similar to the true structure.

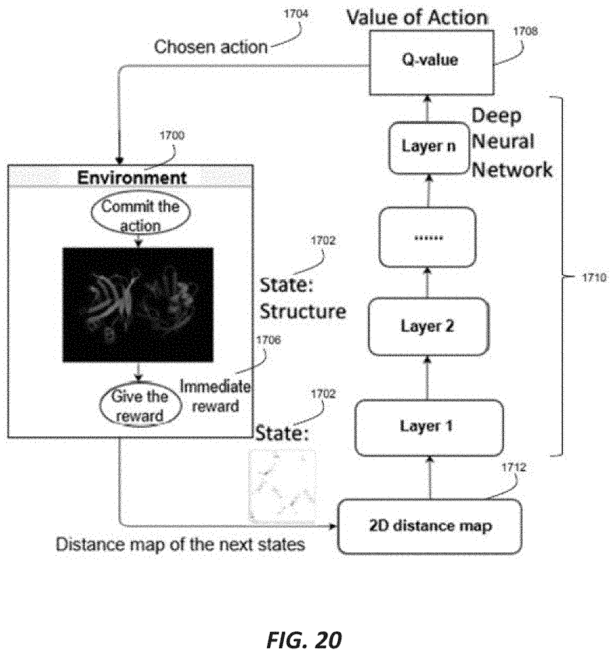

Referring now to FIG. 20, an example approach for DRL to predict a quaternary structure of a protein complex is shown; but is should be understood that the same techniques can be used for predicting the 3D structure of a protein-ligand complex. The DRL of FIG. 20 comprises an AI agent (e.g., learning agent), an environment 1700, a state 1702 (e.g., structure and distance map), an action 1704 (e.g., three rotations and three translations such as described above in connection with the 3-axis coordinates), an immediate reward 1706, a long-term award, a value function 1708 (which can be referred to as Q(s,a), a Q function, and a Q-value), a deep neural network (DNN) 1710, an approximation of the Q function 1708, and policy.

The environment 1700 is used to describe the physical environment that a protein complex or a protein-ligand complex lives in. It describes the physical interactions between proteins, and it provides tools to change the conformation (quaternary structures) of protein complex upon the instructions of the (AI) agent.

The Q-value 1708 is the value of each action predicted by the deep learning network 1710 for an input state. The Q-value is related to the outcome of an action if it is applied. The predicted Q-value tells the reinforcement learning agent which action to take to increase its immediate and long-term reward by taking the action to change the state (e.g., quaternary structure). The higher the Q-value for an action, the more likely it is selected by the agent to adjust the state.

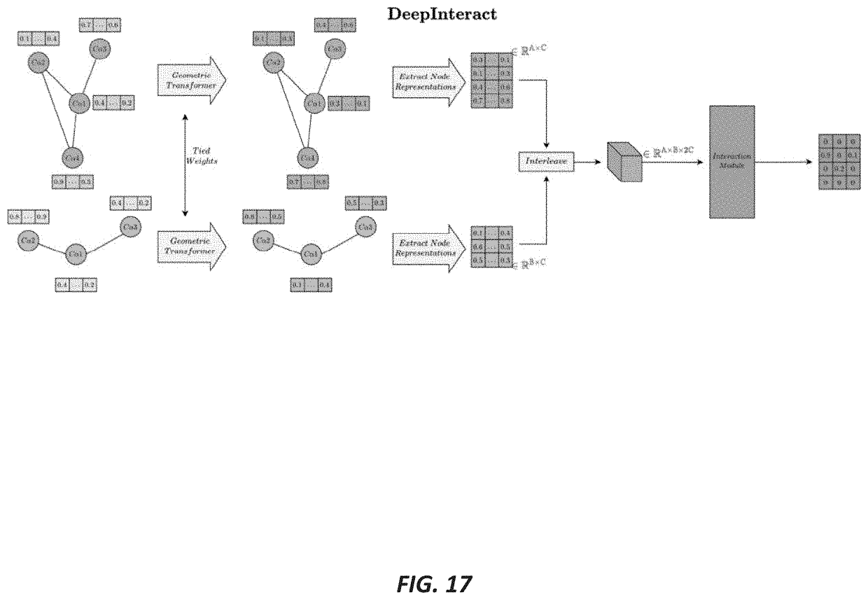

The number of layers (n) of a deep neural network 1710 is called the depth. There is an assumption made that the layers of a deep neural network (e.g., such as DNN 1710)—denoted as Layer 1, Layer 2, . . . , Layer i-1, Layer i, Layer i+1, . . . , Layer n, any Layer i (i>1)—use the output of its previous layer (Layer i-1) as input to generate some hidden features, which are used as input for its following layer (Layer i+1). The first layer (e.g., Layer 1) can be an input layer to take in the input features. The last layer (e.g., Layer n) can be an output layer to generate the final output. The layers in between can be referred to as hidden layers, and the hidden layers generate intermediate hidden features from previous layers. Each hidden layer may take different forms such as a convolutional layer, fully-connected dense layer, attention layer, and normalization layer. For example, FIG. 17 depicts Layer 1, Layer 2 . . . Layer n of DNN 1710.

A 2D distance map 1712 can be used as part of the model, and the 2D distance map 1712 describes the expected physical distance between any two amino acids from the two protein chains (e.g., in the quaternary implementation). This map 1712 can employ the predicted output from the inter-chain distances map produced using the techniques of FIGS. 1-2. The distance tells the optimization methods to position the two amino acids in a way such that their actual distance in the quaternary structure is equal to or close to their distance in the distance map. Therefore, the 2D distance map provides key information to guide the prediction of the quaternary structure. For example, in general terms, if the distances between three points are given, the shape of the three points (e.g., a triangle) will be uniquely defined. So, if a distance map includes sufficiently accurate distances information between amino acids, a quaternary structure can be predicted more accurately with such information.

The deep reinforcement learning (DRL) is trained by a self-learning process. Starting from a randomly initialized structure, the DRL agent takes some actions to change the structure. If the changes are determined to make the structure better (e.g., satisfy more residue-residue contacts or distances), the action will receive some positive rewards, or it will otherwise receive negative rewards. The states (e.g., structures), actions, and rewards generated in this process are used to train the DNN 1710 to predict the Q-value 1708 (e.g., which comprises a summary of both short-term and long-term rewards) of an action for a state. This process is repeated many times until the DNN 1710 can predict the Q-value 1708 of an action for a state to an acceptable level. Once the Q-value 1708 can be accurately predicted, it can be used to guide the agent to select desired actions for states to change the quaternary structure into a structure close to the true structure.

As described herein, the deep learning model, which is deployed in inference mode in FIG. 2, is trained to map the model input (e.g., the data associated with the protein-related complex) to the model output (e.g., the inter-chain distance map for the protein-related complex). The deep learning model is trained using a dataset (or data set), where the dataset includes known inter-chain parameters for a plurality of known protein-related complexes. During deep learning model training, the samples in the dataset are input into the deep learning model, predictions are compared with ground truth, and weights of the deep learning model are adjusted to maximize or minimize an objective function based on errors between predictions and ground truth. For example, as described above, the deep learning model is configured to predict an inter-chain distance map for the protein-related complex based on data associated with the protein-related complex. Thus, for each sample or batch of samples in the dataset, the predicted inter-chain distance maps are compared with their true counterparts to calculate the cross-entropy loss to adjust the weights during training. This training process is repeated until the deep learning model performs with sufficient accuracy. Deep learning model training is well known in the art and also described in further detail in the Examples, such as Example 1.

The dataset can include data that describes a plurality of known protein-related complexes, including known inter-chain distances and/or inter-chain contacts for the known protein-related complexes. The dataset can be stored in one or more sources such as the Protein Data Bank (PDB). As an example, each known protein complex can include a first protein and a second protein, where the first protein comprises a chain of residues and where the second protein comprises a chain of protein residues. These known protein complexes may be protein homodimers where the first and second proteins are copies of each other (e.g., they are monomers that exhibits the same chain of residues) or heterodimers where the two chains are different. In this implementation, inter-chain parameters include a plurality of residue-to-residue distances and/or a plurality of residue-to-residue contacts between residues of the first and second proteins. Alternatively, as another example, each known protein-ligand complex can include a protein and a ligand. In this implementation, inter-chain parameters include a plurality of atom-to-atom distances and/or a plurality of atom-to-atom contacts between atoms of the protein and the ligand.

One or more features can be derived from the dataset. Examples of features that can be derived from the dataset include one or more of the following in any combination of information relating to aspects such as: multiple sequence alignments (MSAs); residue-residue co-evolutionary features of proteins describing the level of the correlated mutations between any two residues of proteins during evolution; structural features (e.g., secondary structures and solvent accessibility of residues); coordinates of residues; residue conservation scores calculated from MSAs; intra-chain contacts; intra-chain distances; inter-chain contacts; and/or inter-chain distances. This disclosure contemplates that features can be converted into numerical values describing the relationships between any two residues between two proteins in a dimer by feature extraction programs or deep learning feature embedding techniques. Such features can be stored in a tensor. It should be understood that the features described above are provided only as examples. For example, in some implementations, the features derived from the dataset include information pertaining to multiple sequence alignments (MSAs), the number of dimers, the length of the dimers (e.g., the length of the monomer sequence in a homodimer), the true inter-chain contacts, and/or true inter-chain distances. It should be understood that the true inter-chain contacts/distances serve as the labels. For a homodimer in which the length of the monomer sequence is L, the input is an L×L×d tensor (e.g., the input features are stored in an L×L×d tensor(s), where d is the number of features for each pair of inter-chain residues).

As described above, the dataset may include known data including various protein and/or ligand data, for example protein complex data with known inter-chain distances/contacts and protein-ligand complex data with known inter-atom distances/contacts. In each case, the dataset can be entered as training/validation (“T/V”) data to train/validate a deep learning model. It should be understood that training/validation (and optionally testing) occurs before the deep learning model is deployed for inference mode (see e.g., FIG. 2). As described herein, a deep learning model includes, for example, an input layer, multiple hidden layers (e.g., with a certain depth), and an output layer. This general framework can be used in the example implementations: (1) for the prediction of inter-protein distances; and (2) for the prediction of inter-atom distances for protein-ligand complexes. The predicted distances can be used by the deep reinforcement learning method or the optimization method such as the gradient descent method as restraints for (1) the prediction of quaternary structures of protein complexes comprising of two or multiple protein chains and for (2) the prediction of 3D structures of protein-ligand complexes.

The input into the model can be sequences and/or tertiary structures of two proteins (e.g., a Chain A and a Chain B), but the data source for the input may include any desired training data in the form of any source of (e.g., protein) data. Such training data may include hundreds of thousands of dimers (e.g., interacting protein pairs), tens of millions of residue-residue pairs, etc. If training data is insufficient, transfer learning can be applied if needed. For example, in some implementations, the dataset may include an open access digital data resource that provides (open) access to biological and medical data for use as experimental data for scientific discovery. Various entities can deposit data in the database so that the data in database can accumulate over time and grow to expand the overall usable dataset present in database. Such continuous growth of the dataset improves training/validating of models. One such example of the database being the Protein Data Bank (“PDB”). The dataset may include structure data for biological molecules (e.g., proteins, DNA, RNA, etc.) from the PDB. For example, as of Apr. 7, 2020, the PDB included 150,672 protein structures, and 81,017 complexes (58,620 homomers, 22,397 heteromers). The more diverse and varied the source data, the better the deep learning can learn and be trained and ultimately the better it can predict.

Although an attention-powered residual neural network is provided as an example deep learning model for predicting inter-chain distances, this disclosure contemplates using other types of deep learning models, For example, in an alternative implementation, the deep learning model deployed in inference mode in FIG. 2 includes a dilated convolutional residual neural network, which is described in detail in Example 2. In this implementation, the model input is the data associated with the protein-related complex includes a structural feature, and the model output is an inter-chain contact map. As described herein, an inter-contact map (i.e., a binary representation) can be derived from the inter-chain distance map.

In yet another alternative implementation, the deep learning model deployed in inference mode in FIG. 2 includes a plurality of convolutional neural networks (CNNs), which is described in detail in Example 3. In this implementation, the model input is the data associated with the protein-related complex includes an MSA-derived feature, and the model output is an inter-chain contact map. As described herein, an inter-contact map (i.e., a binary representation) can be derived from the inter-chain distance map.

In yet another alternative implementation, the deep learning model deployed in inference mode in FIG. 2 includes a graph neural network (GNN), which is described in detail in Example 4. In this implementation, the model input is the data associated with the protein-related complex includes a structural feature, and the model output is an inter-chain contact map. As described herein, an inter-contact map (i.e., a binary representation) can be derived from the inter-chain distance map.

Predicting 3D Structure of Protein-Related Complexes

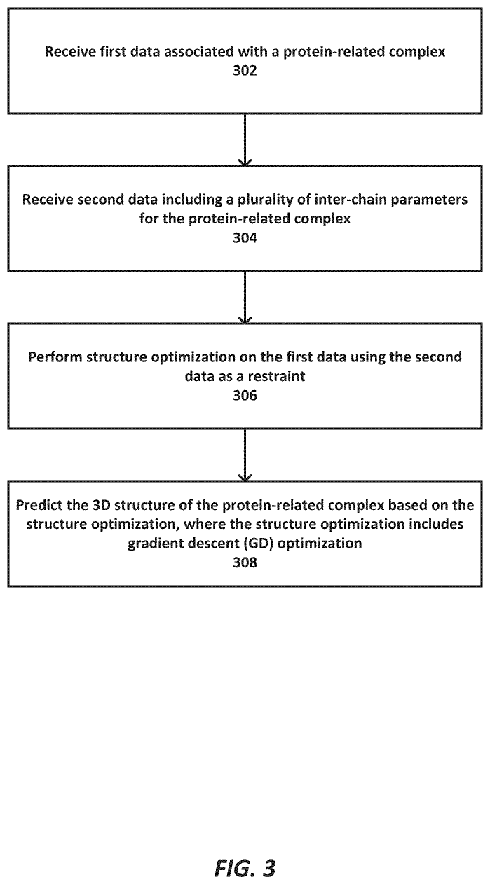

Systems and methods for predicting a three-dimensional (3D) structure of protein-related complexes are described herein. Referring now to FIG. 3, a method for predicting a three-dimensional (3D) structure of protein-related complexes is shown. The predicted 3D structure can be applied to study, engineer and design proteins and protein-related complexes to improve and modulate protein function in many domains including but not limited to medicine, drug design, protein design, agriculture, bioenergy production, and biotechnology development. This disclosure contemplates that the logical operations can be performed using at least one processor and memory (see e.g., computing device 400 shown in FIG. 4). As examples, the protein complex structure prediction techniques described herein can be used in the context of biological renewable energy systems in order to improve the knowledge base for biomass and biodiesel production. Protein complex structure determines various critical biological functions such as carbon assimilation and lipid synthesis. The methods used to predict the structure of protein complexes comprised of multiple proteins as disclosed herein can be applied to proteins of green algae. Each cell of green algae is effectively a tiny ethanol factory, and such (algae) energy can be utilized in bio-renewable energy systems. Conventionally, accurate prediction of such protein complex structures has been difficult to obtain. However, the techniques described herein are expected to exhibit 20% or more accuracy than existing (e.g., protein docking) methods. The impact of this improvement over conventional techniques is expected to provide for a much-needed tool for use in the studying, designing, and engineering of biological renewable energy systems such as those using green algae, in order to improve biomass and biodiesel production.

At step 302, first data associated with a protein-related complex is received, for example, by a computing device. As described herein, in some implementations, a protein-related complex is a protein complex including a first protein and a second protein, where the first protein includes a first chain of residues and the second protein includes a second chain of residues. Optionally, the protein-related complex is a homodimer (i.e., formed by two identical proteins). Alternatively, the protein-related complex is a heterodimer (i.e., formed by two identical proteins). It should be understood that a protein-related complex including two proteins (i.e., homodimers and heterodimers) is provided only as an example. This disclosure contemplates that the protein-related complex may include more than two proteins. In other implementations, the protein-related complex is a protein-ligand complex including a protein and a ligand. It should be understood that a protein-related complex including a protein and a ligand is provided only as an example. This disclosure contemplates that the protein-related complex may include more than one protein and more than one ligand. As described herein, the first data can include information pertaining to multiple sequence alignments (MSAs), the number of dimers, and/or the length of the dimers (e.g., the length of the monomer sequence in a homodimer) in each of the proteins.

At step 304, second data including a plurality of inter-chain parameters for the protein-related complex is received, for example, by a computing device. In implementations where the protein-related complex includes a plurality of proteins (i.e., protein complex), the second data includes a plurality of residue-to-residue distances between residues of the first and second proteins. In other words, the second data may be an inter-chain distance map for the protein-related complex. Alternatively or additionally, the second data includes optionally includes a plurality of residue-to-residue contacts between residues of the first and second proteins. In other words, the second data may be an inter-chain contact map for the protein-related complex. As described herein, the inter-chain distance map for the protein-related complex can be converted into an inter-chain contact map, which is a binary representation of the distances between all possible residue pairs of a protein-related complex.

On the other hand, in implementations where the protein-related complex includes a protein and a ligand (i.e., protein-ligand complex), the second data includes a plurality of atom-to-atom distances between atoms of the protein and the ligand. In other words, the second data may be an inter-chain distance map for the protein-related complex. Alternatively or additionally, the second data includes optionally includes a plurality of atom-to-atom contacts between atoms of the protein and the ligand. In other words, the second data may be an inter-chain contact map for the protein-related complex. As described herein, the inter-chain distance map for the protein-related complex can be converted into an inter-chain contact map, which is a binary representation of the distances between all possible residue pairs of a protein-related complex.

Optionally, the second data may include additional information about the protein-related complex. For example, the second data may optionally include one or more features extracted from: multiple sequence alignments (MSAs); residue-residue co-evolutionary features of proteins describing the level of the correlated mutations between any two residues of proteins during evolution; structural features (e.g., secondary structures and solvent accessibility of residues); coordinates of residues; and/or residue conservation scores calculated from MSAs.

Optionally, in some implementations, the second data is the inter-chain distance map for the protein-related complex as predicted by the deep learning model described above with regard to FIGS. 1 and 2. Alternatively, in other implementations, the second data is the inter-chain distance map for the protein-related complex obtained by other means and/or as found in a database such as the PDB. In other words, the second data need not be predicted using a deep learning model and may instead be based on other conventional techniques.

At step 306, structure optimization is performed on the first data using the second data as a restraint. In particular, the structure optimization includes gradient descent (GD) optimization. GD optimization is described in detail below with regard to Example 5. As described herein, the GD optimization is an iterative optimization process to adjust the positions of protein chains to satisfy the inter-chain contacts, where the inter-chain distance map is as a restraint.

As a result of the structure optimization, at step 308, the 3D structure of the protein-related complex is predicted. Prediction based on the structure optimization is described in detail below with regard to Example 5.

Example Computing Device

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Residue-residue distance information is useful for predicting tertiary structures of protein monomers or quaternary structures of protein complexes. Many deep learning methods have been developed to predict intra-chain residue-residue distances of monomers accurately, but few methods can accurately predict inter-chain residue-residue distances of complexes. Described herein is a deep learning method CDPred (i.e., Complex Distance Prediction) based on the 2D attention-powered residual network to address the gap. Tested on two homodimer datasets, CDPred achieves the precision of 60.94% and 42.93% for top L/5 inter-chain contact predictions (L: length of the monomer in homodimer), respectively, substantially higher than DeepHomo's 37.40% and 23.08% and GLINTER's 48.09% and 36.74%. Tested on the two heterodimer datasets, the top Ls/5 inter-chain contact prediction precision (Ls: length of the shorter monomer in heterodimer) of CDPred is 47.59% and 22.87% respectively, surpassing GLINTER's 23.24% and 13.49%. Moreover, the prediction of CDPred is complementary with that of AlphaFold2-multimer.

Proteins are a key building block of life. The function of a protein is largely determined by its three-dimensional structure 1. Sometimes single-chain proteins (monomers) can perform certain functions, while the structures of most individual proteins interact to form multi-chain complex structures (multimers) to carry out their biological function². Therefore, modeling the three-dimensional structure of both monomers and protein complexes is crucial for studying protein function.

Deep learning has been applied to advance the prediction of the tertiary structures of monomers since 2012³. Over a decade, many deep learning methods were developed to predict intra-chain residue-residue contact maps or distance maps of monomers⁴⁻⁸, which were used by contact/distance-based modeling methods such as CONFOLD⁹ and Rosetta¹⁰ to build their tertiary structures. Extensive studies^{9,11-13} have shown that if a sufficiently accurate intra-chain distance map is predicted, then the protein's tertiary structure can be accurately constructed. Most recently, AlphaFold2¹⁴ uses an end-to-end deep learning method to predict both tertiary structures and residue-residue distances of monomers, achieved a very high average accuracy (^{˜}90 Global Distance Test (GDT-TS) score¹⁵ in the 14th Critical Assessment of Techniques for Protein Structure Prediction (CASP14) in 2020. Recently, AlphaFold2 was extended to AlphaFold-multimer¹⁶ and AF2Complex¹⁷ to improve the prediction of quaternary structures of multimers.

Following the deep learning revolution in the prediction of intra-chain residue-residue distances and tertiary structures, recently some deep learning methods were developed to predict the inter-chain residue-residue contact map of homodimers and/or heterodimers, such as ComplexContact¹⁸, DeepHomo¹⁹, DRcon²⁰, and GLINTER²¹ that predicts the contact map for both homodimers and heterodimers using as input a graph representation of protein monomer structure and the row attention maps generated from multiple sequence alignments (MSAs) by the MSA transformer²². The attention map calculated by the MSA transformer is a kind of residue-residue co-evolutionary feature extracted from MSAs. It has been automatically trained on millions of the MSAs to capture the co-evolutionary information across many diverse protein families during its unsupervised pretraining. Despite the significant progress, the accuracy of inter-chain contact prediction is still much lower than that of intra-chain contact/distance prediction, which calls for the development of more methods to tackle this problem.

A protein complex distance prediction method (CDPred) based on a deep learning architecture combining the strengths of the deep residual network²³, a channel-wise attention mechanism, and a spatial-wise attention mechanism to predict the inter-chain distance maps of both homodimers and heterodimers is described below. As in GLINTER, the attention map of the MSA generated by the MSA transformer is used as one input for CDPred. The predicted distance map for monomers in dimers is used as another input feature. Different from the existing deep learning methods, CDPred predicts inter-chain distances rather than binary inter-chain contacts (contact or no contact) that the current methods such as DeepHomo and GLINTER predict. We test the CDPred rigorously on two homodimer test datasets and two heterodimer test datasets. For these datasets, CDPred yields much higher accuracy than DeepHomo and GLINTER.

Results

Evaluation of Inter-Chain Prediction for Homodimers

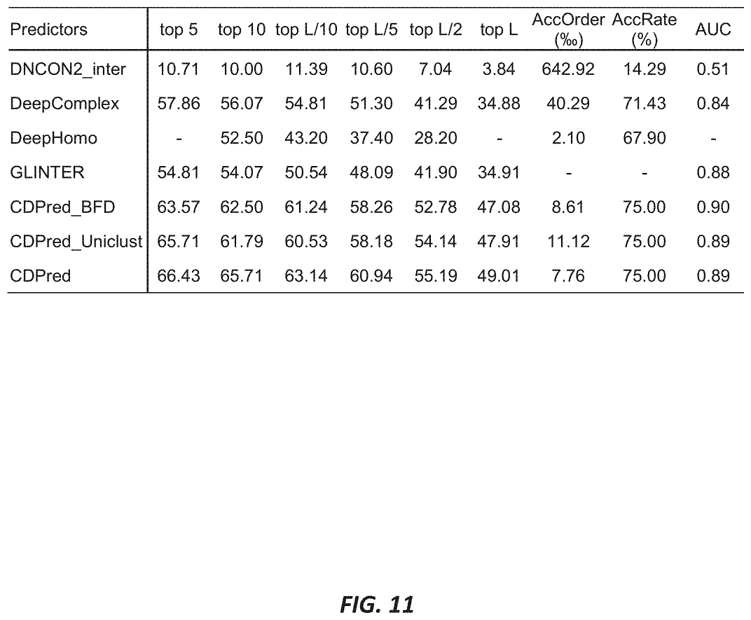

CDPred with DNCON2_inter²⁴, DeepComplex²⁵, DeepHomo, and GLINTER are compared on the HomoTest1 homodimer test dataset with the results shown in Table 1 (FIG. 11). The input tertiary structures for all three methods are predicted structures corresponding to the unbound monomer structures. The DNCON2_inter is run with the recommended parameters. The DeepComplex web server is used to get its prediction results. The results of DeepHomo are obtained from its publication. Three versions of CDPred are tested. The first version (CDPred_BFD) uses the MSAs generated from the BFD database as input. The second version (CDPred_Uniclust) uses the MSAs generated from the Uniclust30 database as input. The third version (CDPred) uses the average of the distance maps predicted by CDPred_BFD and CDPred_Uniclust as the prediction. Because DeepHomo and GLINTER predict binary inter-chain contacts at an 8 angstrom (Å) threshold instead of distances, we convert the inter-chain distance predictions of CDPred, CDPred_BFD, and CDPred_Uniclust into binary contact predictions for comparison. The definition of inter-chain contact is the same as GLINTER and DeepHomo, i.e., a pair of inter-chain residues is considered to be in contact if the distance between their two closest heavy atoms less than 8 Å. This definition is used to evaluate all the inter-chain contact predictions in this work.

CDPred achieves the highest contact prediction precision across the board among all the methods. For instance, CDPred has a top L/5 contact prediction precision of 60.94%, which is 50.34% percentage points higher than DNCON2_inter, 9.64% percentage points higher than DeepComplex, 23.54% percentage points higher than DeepHomo, and 12.85% percentage points higher than GLINTER. CDPred performs better than DNCON2_inter, DeepComplex, DeepHomo, and GLINTER also in terms of Accuracy Rate and AUC score and second best in terms of Accuracy Order. According to almost all the evaluation metrics, CDPred performs better than both CDPred_BFD and CDPred_Uniclust, indicating that averaging the distance predictions made from the two kinds of MSAs can improve the prediction accuracy.

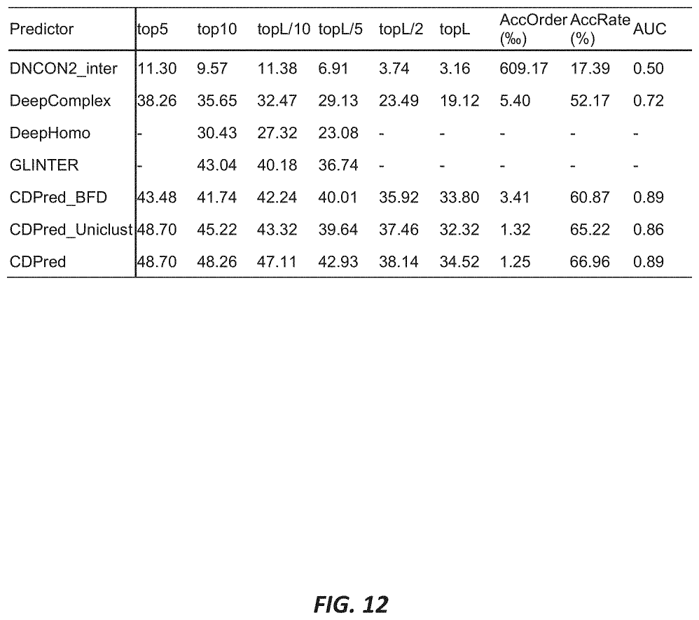

The methods above are also compared on the HomoTest2 homodimer test dataset Table2 (FIG. 12). CDPred performs best in terms of all the evaluation metrics. Combining the predictions of CDPred from two kinds of MSAs improves the prediction accuracy.

The Impact of MSA Depth on the Accuracy of Inter-Chain Contact Prediction for Homodimers

Example 1 shows that two different MSAs (BFD and Uniclust) lead to the different prediction accuracy for CDPred_BFD and CDPred_Uniclust, and CDPred that averages the two contact maps predicted from the two MSAs yields the best result. Here, the depth of MSAs and a direct combination of the two MSAs are investigated to see how they may affect the prediction accuracy. Supplementary data shows the number of sequences and the number of effective sequences (Neff) 2 for each dimer in HomoTest1 and HomoTest2 as well as the top L/2 contact prediction precision of CDPred_BFD, CDPred_Uniclust, CDPred, and CDPred_ComMSA that uses the simple combination of the BFD MSA and Uniclust MSA as input. Neff weights similar sequences in MSA less in counting the number of sequences and is widely used to measure the depth of MSA.

The Neff and contact prediction precision for CDPred_BFD and CDPred_Uniclust vary from target to target. The Pearson correlation coefficient between the difference of Neff and the difference of the top L/2 precision for CDPred_BFD and CDPred_Uniclust is 0.31 and 0.67 on HomoTest1 and HomoTest2, respectively, indicating that the depth of MSA has some positive impact on the contact prediction precision. CDPred_ComMSA which combines the two MSAs to generate a deeper MSA as input, performs better than both CDPred_BFD and CDPred_Uniclust on HomoTest1 and better than CDPred_BFD on HomoTest2, suggesting that directly combining two MSAs can be beneficial. CDPred still performs slightly better than CDPred_ComMSA in terms of top L/2 prediction precision on both datasets (55.19% versus 55.13% on HomoTest1 and 38.14% versus 36.14% on HomoTest2) indicating that averaging the distance maps predicted from the two MSAs is more effective than simply combining the two MSAs as input.

Evaluation of Inter-Chain Contact Predictions for Heterodimers

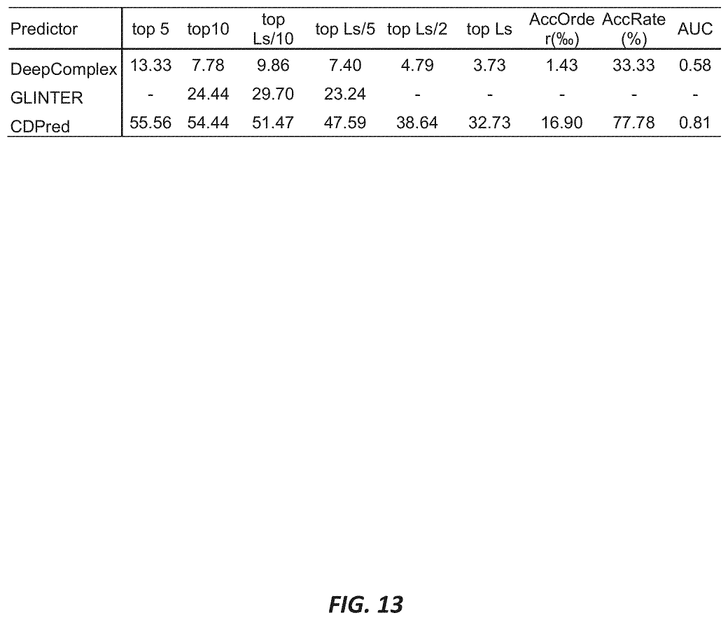

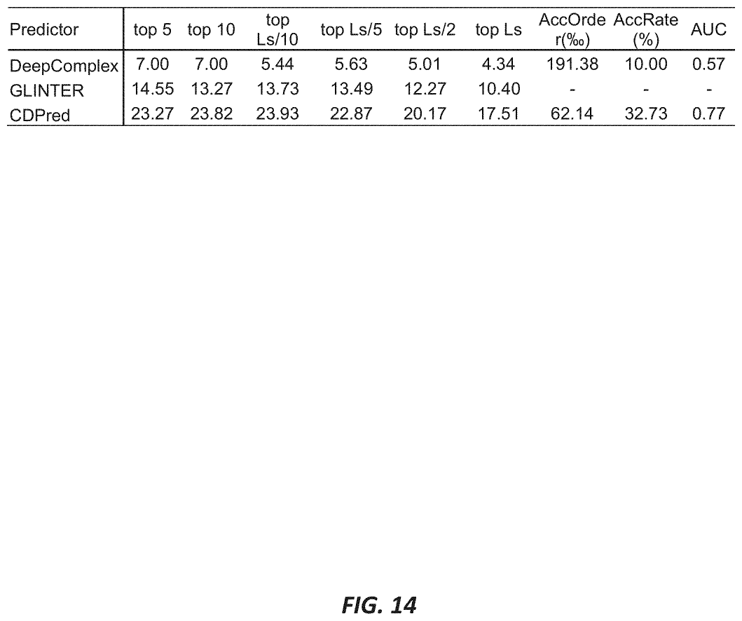

CDPred and a state-of-the-art heterodimer contact predictor GUNTER are compared on both HeteroTest1 and HeteroTest2 heterodimer test datasets (see results in Table 3 (FIG. 13) and Table 4 (FIG. 14), respectively). The input tertiary structures of monomers used by both methods are predicted by AlphaFold2. Two different orders of monomer A and monomer B (AB and BA) are used in each heterodimer to generate input features for CDPred to make predictions. The average of the outputs of the two orders is used as the final prediction. The inter-chain part of the BA prediction map is taken out and transposed to the same shape as its counterpart in the AB prediction map before they are averaged.

On the HeteroTest1 dataset (Table3, FIG. 13), CDPred achieves much better performance than GLINTER in terms of all the metrics. It is also substantially better than DeepComplex in terms all the metrics but Accuracy Order. For instance, the top Ls/5 contact prediction precision of CDPred 47.59% is more than twice 23.24% that of GLINTER, and 40.19% percentage points higher than DeepComplex. On the HeteroTest2 dataset (Table 4, FIG. 14), CDPred also substantially outperforms DeepComplex and GLINTER in terms of all the metrics (contact precisions, Accuracy Order, Accurate Rate, and/or AUC).

Supplemental data compares the performance of using the two different orders of monomers as input (CDPred(A_B) and CDPred(B_A)) and averaging the outputs of the two different orders (CDPred) on the HeteroTest1 and HeteroTest2 datasets, respectively. The accuracy of CDPred(A_B) and CDPred(B_A) varies from target to target and from dataset to dataset. Sometime the precision of the two orders can be substantially different. However, a two-sided pairwise t-test shows that there is no significant difference between the two on average. Even though averaging the contact maps predicted in two different orders does not always yield the best accuracy, it makes the performance more stable by reducing the variance and smoothing the prediction. For instance, CDPred often delivers either the best or medium prediction accuracy in comparison with CDPred(A_B) and CDPred(B_A).

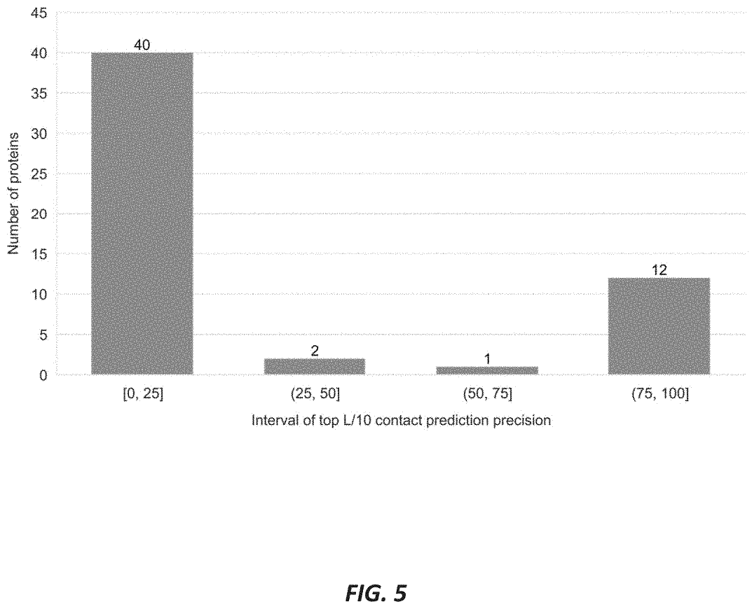

Furthermore, the top L/10 contact prediction precisions for the heterodimers in the more challenging HeteroTest2 dataset are divided into four equal intervals and plot the number of heterodimers in each interval (see FIG. 5). The precision of the predictions in the four internals is bifurcated, mainly centered on a low precision interval [0%-25%] and a high precision interval [75%-100%]. 40 heterodimers have low contact prediction precision in the range of 0%-25%, indicating there is still a large room for improvement. One reason for the low precision is that most of the 40 heterodimers have shallow MSAs. The Pearson correlation coefficient between the logarithm of the number of effective sequences (Neff) of MSA and the top L/10 complex contact precision is 0.46, indicating a modest correlation between the two.

It is also observed that the inter-chain contact prediction accuracy for heterodimers is lower than for homodimers on average. One reason is that the MSA generation for a homodimer only needs to generate an MSA for a monomer in the homodimer, which is usually much deeper than the MSA generated for a heterodimer that requires the pairing of the related sequences in the MSAs of two different monomers in the heterodimer. Another reason is that homodimers tend to have a larger interaction interface than heterodimers on average, making the prediction easier.

Comparison of the Co-Evolutionary Features Generated by the Statistical Optimization Method and Deep Learning Method

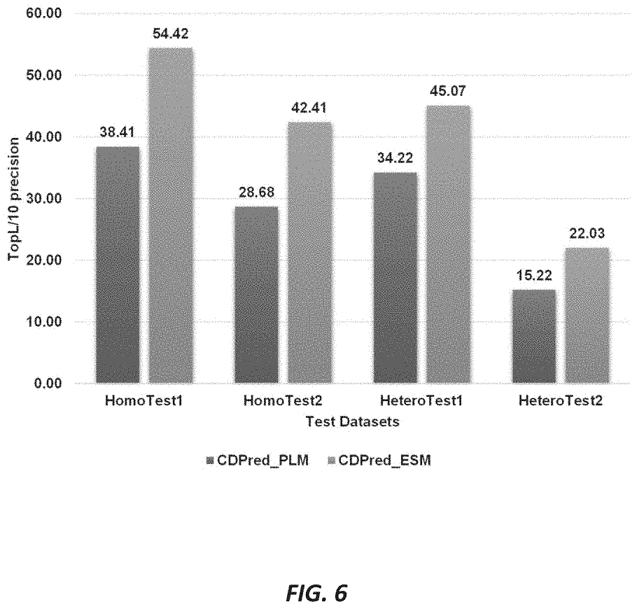

To compare the performance of the co-evolutionary feature generated by the statistical optimization tool—CCMPred and the deep learning tool—MSA transformer, two different models are trained on the two different kinds of co-evolutionary features of the same training dataset using the same neural network architecture. One network (CDPred_PLM) is trained on the PLM co-evolutionary features generated by CCMPred. Another one (CDPred_ESM) is trained on the row attention map features generated by the MSA transformer. The precision of the top L/10 contact predictions of the two models on the four different test datasets are plotted in FIG. 6. CDPred_ESM has better performance than CDPred_PLM on all the four test datasets, indicating that the co-evolutionary features extracted automatically by the deep learning method is more informative than by the statistical optimization method of maximizing direct co-evolutionary signals. However, combining the two kinds of co-evolutionary features yields even better results (see the results in Tables 1, 2, 3, and 4). Supplemental data shows the top L/10 precision of CDPred_ESM against the top L/10 precision of CDPred_PLM for the homodimers in the two homodimer test datasets and the heterodimers in the two heterodimers test datasets, respectively. For 42 out of 51 homodimers and 55 out of 64 heterodimers, CDPred_ESM has higher precision than CDPred_PLM. Both CDPred_ESM and CDPred_PLM can perform better on some targets, indicate the co-evolutionary features used by the two methods have some complementarity.

The Impact of the Quality of Predicted Tertiary Structures on Monomers on Inter-Chain Distance Prediction of Dimers

The quaternary structure of a protein complex depends on the tertiary structure of its monomer units. As Alphafold can predict the tertiary structure of monomers very well, it is investigated how effectively AlphaFold-predicted tertiary structures can be applied to predict inter-chain distance maps for protein complexes. The TM-scores of the predicted tertiary structure for each monomer unit of each dimer and the contact prediction precision of CDPred on the four datasets (HomoTest1, HomoTest2, HeteroTest1, and HeteroTest2) are shown in supplemental data. The average TM-scores of the predicted tertiary structures for HomoTest1 and HomoTest2 are 0.95 and 0.90, for Chain A of heterodimers in HeteroTest1 and HeteroTest2 are 0.90 and 0.89, and for Chain B of heterodimers in HeteroTest1 and HeteroTest2 are 0.95 and 0.88, respectively, indicating the AlphaFold predicted tertiary structures have high quality. The Pearson's correlation between the TM-score of the predicted tertiary structures and top L/2 contact prediction precision is 0.19. The weak correlation may be partly due to that the quality of predicted tertiary structures is high enough in general for CDPred to leverage most tertiary structure information to predict inter-chain distances.

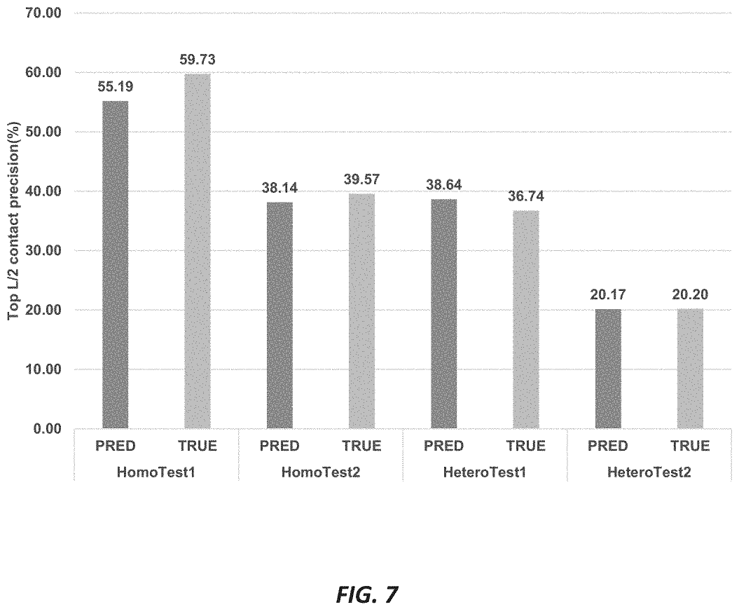

Moreover, the top L/2 inter-chain contact prediction precision of using AlphaFold predicted tertiary structures of monomers as input and using true tertiary structures of monomers in the bound state as input are compared on the four datasets (FIG. 7). Using the true tertiary structures yields slightly better performance than using the AlphaFold predicted structures on three out of four datasets (HomoTest1, HomoTest2 and HeteroTest2), but slightly worse performance on HeteroTest1. The p-value of the pair-wise t-test of the difference on the four datasets is 0.6802, 0.8892, 0.9083, and 0.9963, respectively, indicating that the difference is not significant. The results show that the AlphaFold-predicted tertiary structures are sufficiently accurate for CDPred to make inter-chain distance prediction, even though using true tertiary structures as input can slightly improve the prediction accuracy overall. This is different from GiLNTER whose accuracy of using true tertiary structures as input is substantially higher than using AlphaFold predicted tertiary structures as input²¹.

High Correlation Between the Precision of Inter-Chain Contact Predictions and Predicted Probability Scores

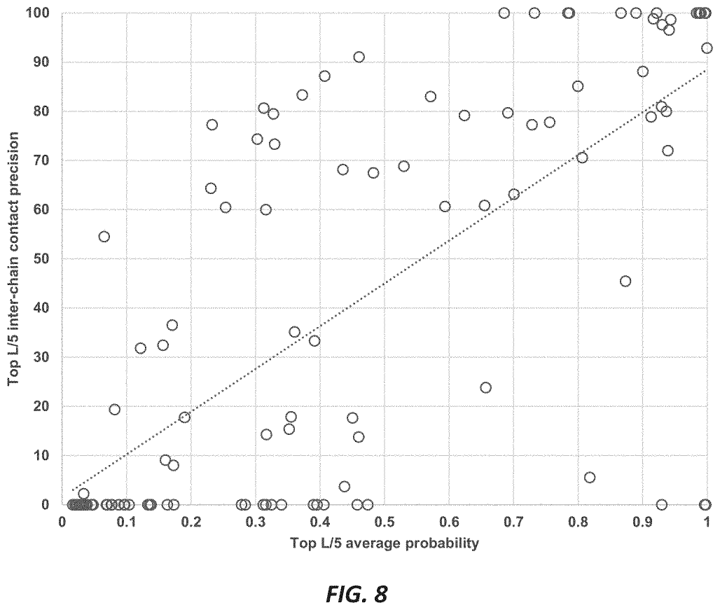

The previous work on the intra-chain distance prediction²⁷ shows that the intra-chain distance prediction accuracy and predicted probability scores have a strong correlation, which can be used to select predicted intra-chain distance maps. Here, it is investigated if the similar correlation exists in the inter-chain distance prediction. FIG. 8 is a plot of the precision of top L/5 inter-chain contact predictions and the average of their probability scores for each target in the four test datasets. The correlation between the top L/5 inter-chain contact precision and the average predicted probability score is 0.7345. The high correlation suggests that the probability of inter-chain contacts predicted by CDPred can be used to estimate the confidence of the inter-chain prediction.

The Comparison Between CDPred and AlphaFold2-Multimer

AlphaFold2-multimer is currently the-state-of-the-art method for predicting quaternary structures of multimers. To investigate if CDPred is complementary with AlphaFold2-multimer, their inter-chain contact prediction accuracy is compared on the four datasets. The comparison is not completely fair because the redundancy between the test datasets and AlphaFold2-multimer's training dataset is not removed. The latest version (Version 2) of AlphaFold2-multimer without templates was run to predict the quaternary structures for the dimers in the four test datasets. The inter-chain distance maps are extracted from the predicted quaternary structures. Each distance in the map is inverted to generate a contact probability map to be compared with the inter-chain contact map predicted by CDPred. Supplemental data presents a target-by-target comparison of top L/2 inter-chain contact prediction precision of CDPred and AlphaFold2-multimer for each target in the four test datasets. AlphaFold2-multimer has higher top L/2 precision than CDPred on the majority of the targets. However, for the very hard 44 targets on which the top L/2 precision of AlphaFold2-multimer is less than 10%, CDPred performs better than AlphaFold2-multimer on 15 targets, equally on 25 targets, and worse on 4 targets. On the 19 hard targets that the two methods perform differently, the average precision of CDpred is 14.8%, much higher than 1.79% of AlphaFold2-multimer. The p-value of the two-sided pairwise t-test of the difference is 0.0068, indicating it is significant. For instance, for target 7LB6, the top L/2 precision of CDPred is 44.62%, much higher than 0% of AlphaFold2-multimer. The Neff of the MSA of the target is 16.6. The results show that CDPred is complementary with AlphaFold2-multimer and can be particularly useful when the target is very hard and AlphFold2-multimer prediction has very low confidence. One possible application of CDPred is to use its predicted distance map to rank and select diverse quaternary structural models of hard targets predicted by AlphaFold2-multimer.

An Inter-Chain Distance Prediction Example

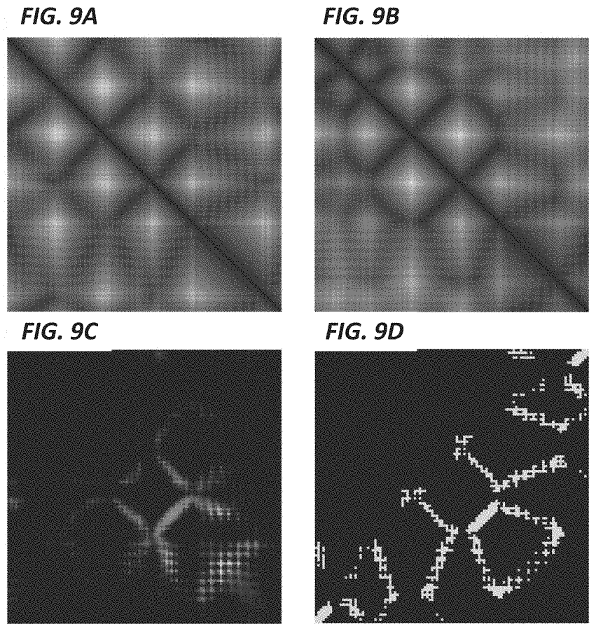

Typically, when the MSA is shallow, the precision of inter-chain distance prediction is low due to the lack of information. However, CDPred still can accurately predict inter-chain distance for some targets with shallow MSAs. FIGS. 9A-9D show such a CASP13 homodimer target T0991, the distance map is visualized by matplotlib²⁸. Its MSA has only one sequence. The TM-score²⁹ of the tertiary structure of the monomer of T0991 predicted by AlphaFold2 is 0.3104, indicating the predicted tertiary structure fold is not correct. However, the precision of top L/10, top L/5, and top L/2 inter-chain contacts derived from the distance map predicted by CDPred is 72.73%, 68.18%, and 56.36%, respectively, which is high. FIGS. 9A and 9B show the intra-chain distance maps of the AlphaFold predicted tertiary structure and the true tertiary structure of the monomer, FIG. 9C shows the inter-chain contact map predicted by CDPred, and FIG. 9D shows the true inter-chain contact map. The predicted inter-chain contact map accurately recalls a large portion of the true inter-chain contacts.

Methods

Attention-Based Neural Network Architecture

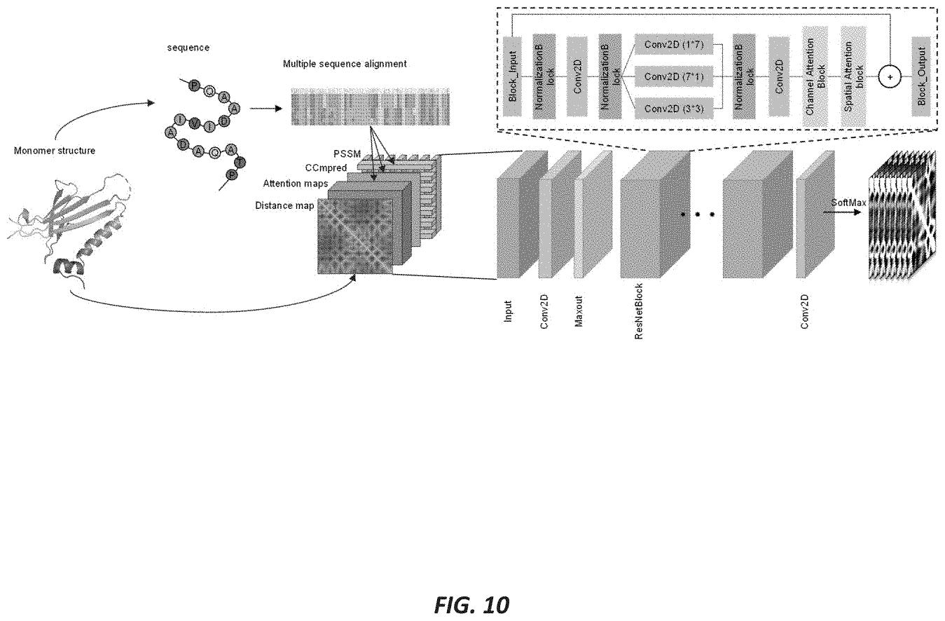

FIG. 10 illustrates the overall architecture of CDPred based on the channel-wise and spatial-wise attention mechanisms. CDPred takes the tertiary structures of monomers of a dimer as input and extracts the monomer sequences and intra-chain distance maps. For homodimers, since the sequences of the two monomers of a homodimer are the same, only one monomer tertiary structure is used as input. The monomer sequences are used to search the protein sequence databases to generate MSAs of dimers, which are used to generate residue-residue co-evolutionary scores, row attention maps, and position-specific scoring matrix (PSSM) as input features. The complete input for CDPred is the concatenation of all the input features.

The input features stored in 2D tensors of multiple channels are first transformed by a 2D convolutional layer, followed by a Maxout layer³⁰ to reduce the dimensionality. The output of the Maxout layer is used as input for a series of deep residual network blocks empowered by the attention mechanism. The residual network has been widely used in computer vision and protein intra-chain distance and contact prediction^{5,7,31}. Here, the residual connection is combined with other useful components to construct a residual block, which includes the normalization block (called RCIN) consisting of row normalization layer (RN), column normalization layer (IN)³², and instance normalization (IN)³³ for normalizing the feature maps, a channel attention squeeze-and-excitation (SE) block³⁴ for capturing the important information of different feature channels, and a spatial attention block³⁵ that captures signals between residues right after the channel attention block. Following the residual blocks, a 2D convolutional layer with the softmax function is used to classify the distance between any two residues from two monomers in a dimer into 42 distance bins (i.e., 40 bins from 2 to 22 Å with a bin size of 0.5 Å, plus a 0-2 Å bin and a >22 Å bin). Two kinds of inter-chain residue-residue distance are predicted at the same time: (1) the distance between the two closest heavy atoms from two residues used by most existing works in the field and (2) the C_{b}-Cₑ distance between two residues used by some recent works³⁶, resulting in two kinds of distance maps predicted.

Features

The input features of CDPred contain (1) the tertiary structure information of monomers in the form of intra-chain distance map, (2) pair-wise co-evolutionary features, and (3) sequential amino acid conservation features, which are stored in a L×L×N tensor (L is the length of the sequence of a monomer for a homodimer or the sum of the length of two monomers (L1+L2) for a heterodimer). N is the number of feature channels for each pair of residues.

Tertiary structure information of monomers: The protein tertiary structure information of a monomer in a dimer is represented as an intra-chain distance map storing the distance between Ca atoms of two residues in the monomer. For a homodimer, an intra-chain distance map (L×L×1) computed from the tertiary structure of only one monomer is used. For a heterodimer, two intra-chain distance maps (L1×L1×1 and L2×L2×1) of the two monomers in the heterodimer are computed from their tertiary structures and added as the top left submatrix and the bottom right submatrix of the input distance map of the dimer of (L+L2)×(L1+L2)×1 dimension. The values of the other area of the input distance map of the heterodimer are set to 0. In the training phase, the true tertiary structures of monomers in the dimers are used to compute the intra-chain distance maps above. During the test/prediction phase, the tertiary structures of monomers predicted by AlphaFold are used to generate the intra-chain distance maps as input. Using predicted tertiary structures as input is more challenging but can more objectively evaluate the performance of inter-chain distance prediction because in most situations the true tertiary structures of the monomers are not known. A predicted tertiary structure also corresponds to an unbound tertiary structure, a term commonly used in the protein docking field.

Co-evolutionary features: MSAs are generated for homodimers or heterodimers as input for the calculation of their co-evolutionary features. To challenge the deep learning method to effectively predict inter-chain distance maps from noisy inputs, in the training phase, the less sensitive tools or smaller sequence databases are used to generate MSAs, but in the test phase, state-of-the-art tools and larger databases are used to generate the requisite MSAs. Specifically, in the training phase, for a homodimer, PSI-BLAST³⁷ is used to search the sequence of a monomer against Uniref90 (2018-04)³⁸ to generate the MSAs, and for a heterodimer, the procedure in FoldDock³⁹ using the HHblits⁴⁰ is used to search against Uniclust30 (2017-10) to generate the MSA for each of the two monomers and then pair the two MSAs to produce an MSA for the heterodimer according to the organism taxonomy ID of the sequences.

In the test stage, for a homodimer, HHblits is used to search the sequence of a monomer against the Big Fantastic Database (BFD)⁴¹ and Uniclust30 (2017-10) respectively to generate two MSAs for a single chain of the homodimers, which are used to generate input features separately to make two predictions that are averaged as the final predicted distance map; for a heterodimer, an MSA is generated by the same procedure used in EvComplex2⁴², which applies the jackhammer to search against Uniref90 (2018-04) to generate one MSA for each of the two monomers and then pairs the sequences from the two MSAs to produce an MSA for the heterodimer according to the highest sequence identity with the monomer sequences in each species. The MSA for a homodimer or a heterodimer is used by a statistical optimization tool CCMpred⁴³ to generate a residue-residue co-evolutionary score matrix (L×L×1) as features and by a deep learning tool MSA transformer²² to generate residue-residue relationship (attention) matrices (L×L×144) as features. L is the number of the columns in MSA.

Sequential features: The sequence profile (i.e., position-specific scoring matrix (PSSM)) of protein generated by the PSI-BLAST search above contains the residue conservation information. The PSSM of a monomer in a homodimer or the vertical concatenation of two PSSMs of two monomers in a heterodimer in the shape of L×20 is tiled (i.e., cross concatenated element by element) to generate sequential features of dimensionality L×L×40.

Training Procedure and Hyperparameters

The deep neural network uses the input features above to predict a heavy atom distance map and a C_{b} distance map of shape L×L×42. The 42 channels store the probability of a distance between two residues in 42 distance bins. The predicted inter-chain distance maps are compared with their true counterparts to calculate the cross-entropy loss to adjust the weights during training. For a heterodimer (L=L1+L2), an output distance map of dimension (L1+L2)×(L1+L2)×42 contain both inter-chain distance predictions and intra-chain distance predictions. Only inter-chain distance predictions are used to calculate the cross-entropy loss to train the networks, while the intra-chain distance predictions are ignored. The number of convolutional layers of CDPred is set to 156, and the number of filters of each convolutional layer is set to 64. The batch size in training is set to 1 due to the limitation of GPU memory. The Adam optimizer with an 1e-3 learning rate is used to train the model for the first 30 epochs to achieve the fast convergence and used stochastic gradient descent with the 1e-4 starting learning rate and 10-time reduction every 20 epochs for the remaining 50 epochs to further reduce the training loss.

Datasets and Evaluation Metrics

The DeepHomo training dataset¹⁹ is used to train the homodimer inter-chain distance predictor. The whole dataset includes 4,132 homodimeric proteins with C2 symmetry. And after removing proteins that have >=30% sequence identity with the blind test datasets (HomoTest1 and HomoTest2) consisting of the targets of the CASP/CAPRI experiments using MMseq2⁴⁴, 4129 homodimers are left as training, validation, and internal test data. The same as DeepHomo, we select 300 of them as the validation data and 300 as the internal test data and use the rest as the training data. The test dataset used by DeepHomo that contains 28 targets collected from the CASP10-13 experiments is used as one blind homodimer test dataset (HomoTest1). Another test dataset used by GINTER²¹, which includes 23 homodimer targets collected from the CASP13 and 14, is used as the other blind homodimer test dataset (HomoTest2). The two blind test datasets have six common targets.

For heterodimers, the heterodimers in Apoc⁴⁵ is used to create the training, validation, and internal test datasets. After filtering out similar sequences at the 40% sequence identity threshold and removing the sequences with >=30% sequence identity with the blind test datasets (HeteroTest1 and HeteroTest2), 3955 heterodimers are left. 3576 of them are randomly selected as the training data, 198 as the validation data, and 181 as the internal test data. The test dataset used by GLINTER which contains nine heterodimer targets from the CASP13 and CASP14 experiments in conjunction with the CAPRI experiments is used as a blind test dataset (HeteroTest1). To create a larger blind test dataset, we collect the heterodimer released between 09-2021 and 11-2021 in the PDB. After filtering out similar sequences at a 40% sequence identity threshold and excluding sequences with >1000 residues targets, 55 heterodimers are left to create another blind test dataset (HeteroTest2).

Since the external methods GLINTER and DeepHomo predict inter-chain contacts instead of inter-chain distances, to fairly compare CDPred with them, the precision of contact prediction as the evaluation metric. Specifically, the precision of top 5, 10, L/10 (or Ls/10), L/5 (or Ls/5), L/2 (or Ls/2), and L (or Ls) contact predictions (L: length of a monomer in homodimers, Ls: length of the shorter monomer in heterodimers) is computed and compared. The similar metric is also widely used in evaluating intra-chain contact prediction. Because DeepHomo and GLINTER predict inter-chain contacts at an BA threshold, the same threshold is used to convert the distance maps predicted by CDPred into the binary contact map. A predicted inter-chain contact is correct if the minimal distance between the heavy atoms of the two residues is less than BA. The accuracy order, accuracy rate 4, and AUC score are also used to evaluate the inter-chain distance prediction of CDPred. The accuracy order is the rank of the first correct contact prediction divided by the total number of residues of a dimer. AccRate is the percentage of dimers for which at least one of the top 10 inter-chain contact prediction is correct.

### Example 2

Deep learning has revolutionized protein tertiary structure prediction recently. The cutting-edge deep learning methods such as AlphaFold can predict high-accuracy tertiary structures for most individual protein chains. However, the accuracy of predicting quaternary structures of protein complexes consisting of multiple chains is still relatively low due to lack of advanced deep learning methods in the field. Because interchain residue-residue contacts can be used as distance restraints to guide quaternary structure modeling, described herein is a deep dilated convolutional residual network method (DRCon) to predict interchain residue-residue contacts in homodimers from residue-residue co-evolutionary signals derived from multiple sequence alignments of monomers, intrachain residue-residue contacts of monomers extracted from true/predicted tertiary structures or predicted by deep learning, and other sequence and structural features.

Tested on three homodimer test datasets (Homo_std dataset, DeepHomo dataset and CASP-CAPRI dataset), the precision of DRCon for top L/5 interchain contact predictions (L: length of monomer in a homodimer) is 43.46%, 47.10% and 33.50% respectively at 6 Å contact threshold, which is substantially better than DeepHomo and DNCON2_inter and similar to Glinter. Moreover, our experiments demonstrate that using predicted tertiary structure or intrachain contacts of monomers in the unbound state as input, DRCon still performs well, even though its accuracy is lower than using true tertiary structures in the bound state are used as input. Finally, this example shows that good interchain contact predictions can be used to build high-accuracy quaternary structure models of homodimers.

Datasets

Two residues from the two chains in a homodimer are considered an interchain contact if the Euclidean distance between any two heavy atoms of the two residues is less than or equal to 6 Å (Ovchinnikov et al., 2014; Quadir et al., 2021a,b; Zhou et al., 2018). Multiple homodimer datasets with known quaternary structures and interchain contacts are used to develop DRCon. The Homo_std dataset used in DNCON2_Inter is used to train, validate and test DRCon. Homo_std was derived from the homodimers in the 3D Complex database (Levy et al., 2006). All the complexes of the 3D Complex were released before October of 2005. The dimers in the database whose two chains have 295% sequence identity are treated as homodimers to create Homo_std. Homo_std has 8530 homodimers in total that has 530% pairwise sequence identity. It is split into a training dataset (5975 dimers), a validation dataset (853 dimers) and a test dataset (1702 dimers) according to the ratio of 7:1:2 to train, validate and test DRCon. Furthermore, in addition to the 6 Å threshold, 8 Å is also used as a threshold to generate inter-chain contacts to train a network in order to compare it with two inter-chain contact predictors [DeepHomo and Glinter (Xie and Xu, 2021)] trained at the threshold.

Moreover, two independent datasets (the CASP-CAPRI dataset and DeepHomo dataset) are used to test DRCon. The CASP-CAPRI dataset contains 40 homodimers collected from CASP-CAPRI-11, 12, 13 and 14 experiments that are publicly available. We discarded homodimers whose monomer has more than 500 residues in order to make a fair comparison with Glinter as it has a limitation of about 1024 residues for the combined length of the two monomers in a dimer.

The DeepHomo dataset used here contains 218 homodimers out of the 300 homodimers in its original version (Yan and Huang, 2021). 82 homodimers in the original DeepHomo dataset that are present in the Homo_std training dataset are removed to avoid the evaluation bias.

Input Features

The input features for DRCon are stored in L×L×d tensors (L: length of the sequence of the monomer in a homodimer; d is the number of features for each pair of interchain residues) that describe the features of all pairs of interchain residues. Since the two chains in a homodimer are identical and interchain residue-residue coevolution features are also preserved in the multiple sequence alignment (MSA) of one chain (monomer), only the sequence of a monomer is used to generate the input features for interchain contact prediction in this work.

The number of features (d) for each interchain residue pair is 592. 49 features are the same kind of features used by DNCON2 (Adhikari et al., 2018) for intrachain contact prediction, including solvent accessibility of residues as well as interchain residue-residue coevolution features calculated from MSAs of a monomer by CCMpred (Seemayer et al., 2014) and PSICOV (Jones et al., 2012). 526 features generated from MSAs by trRosetta (Yang et al., 2020) are also used. The 8-state secondary structure prediction for each residue (i.e. 16 features for a pair of residues) made by SCRATCH (Cheng et al., 2005) is also included. Finally, a binary feature indicating if two residues form an intrachain contact (i.e. Cb-Cb atom distance is less than or equal to 8 Å (Adhikari et al., 2015; Wu et al., 2021) is also used as input, which is useful for the neural network to distinguish interchain contacts from intrachain contacts. It worth noting that all the features except secondary structure, solvent accessibility, intrachain contact and CCMpred features used in DRCon are different from DeepHomo. In the training phase, the intrachain contacts are derived from the true tertiary structures of monomers in the dimers. In the test phase, the intrachain contacts may be either derived from true tertiary structures of monomers in the bound state or predicted from sequences/tertiary structure models of monomers in the unbound state, depending on the experimental setting. Specifically, for the training and validation datasets, the intrachain contacts are derived from the known tertiary structures of the monomers in the homodimers (the bound state). For the test datasets, either true intrachain contacts or predicted intrachain contacts made by trRosetta or extracted from AlphaFold2 tertiary structure models in the unbound state are used to generate intrachain contact features.

Most of the 592 features above are generated from the MSAs of the monomers in the homodimers. The DNCON2's MSA generation procedure is used to generate the MSAs for all the datasets by using HHBlits (Remmert et al., 2011) to search UniRef30_2020_02 database (Suzek et al., 2015) and Jackhmmer (Johnson et al., 2010) to search Uniref90. In addition, DeepMSA (Zhang et al., 2020) is used to generate MSAs for the CASP-CAPRI dataset. The MSAs with more sequences generated by DNCON2 or DeepMSA are selected for the proteins in this dataset.

Deep Learning Architecture

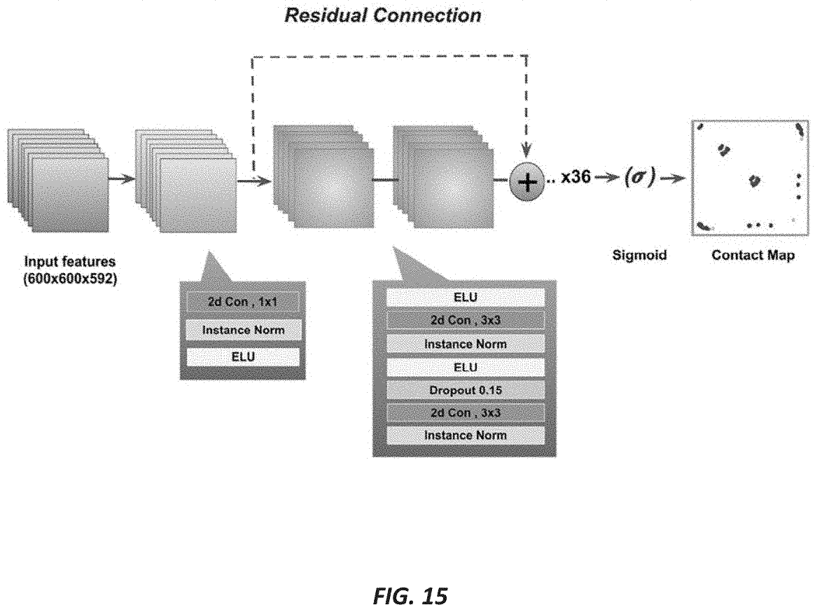

FIG. 15 illustrates the deep learning architecture for interchain contact prediction. The input tensor (L×L×592) is first transformed by a block consisting of a convolutional layer and instance normalization. The instance normalization instead of the batch normalization is used because the former is better at dealing with a small batch size (Lian and Uu, 2019). The transformed tensor is then processed by 36 residual blocks containing regular convolutional layers, instance normalization, dilated convolutional layers and residual connections. The residual connection makes the learning of deep networks more efficient and effective. The dilated convolution can capture a larger input area than the regular convolution with the same number of parameters, which has been shown to improve intrachain residue-residue distance prediction in AlphaFold1 (Senior et al., 2019). This dilated residual architecture is different from that in DeepHomo.

The deep learning architecture of DRCon for interchain contact prediction in homodimers. For a homodimer in which the length of the monomer sequence is L, the input is a L×L×592 tensor. The number of input features for each pair of residues is 592. For convenience, L is set to a fixed number—600. 0 padding is applied if L is less than 600. It is worth noting that in the prediction phase, no zero padding is used in generating the input tensor if L is greater than 600. The input is transformed to a 600×600×48 tensor using a 2D-convolutional layer which has a kernel size of 1 and uses Exponential Linear Unit (elu). The output of the convolution layer is passed through 36 residual blocks with kernel size of 3×3. Each residual block uses a 2D-convolution layer with a kernel size of 3, instance normalization and dropout of 15% probability of a neuron being ignored, followed by a dilated convolution layer without dropout. The step of the dilation in the dilated convolution layers in these blocks changes from 1, 2, 4, 8, 16 periodically. The sigmoid activation function is applied to the output of the last residual block to calculate the contact probability of each interchain residue-residue pair. The probabilities for residue pair (i, j) and residue pair (j, i) are averaged to a symmetric final contact map.

The network is trained on the Homo_std training dataset with 0.0001 learning rate and optimized with Adam (Kingma and Ba, 2015) optimizer using a batch size of 2 and the binary cross entropy as loss function. Each epoch of training the network on six 32 GB NVIDIA V100 GPUs takes around 2 h. The deep network is implemented on Pytorch and horovod (Sergeev and Del Balso, 2018) to leverage the distributed deep learning training. The deep learning model with the highest precision for top L/5 interchain contact predictions on the Homo_std validation dataset is selected as the final model for testing.

Results and discussion of the evaluation of DRCon is described in Appendix A of U.S. provisional patent application No. 63/280,037, filed on Nov. 16, 2021, and titled “Deep Learning Systems and Methods for Predicting Structural Aspects of Protein-Related Complexes,” the disclosure of which is expressly incorporated herein by reference in its entirety.

### Example 3

Significant improvements in the prediction of protein residue-residue contacts are observed in the recent years. These contacts, predicted using a variety of coevolution-based and machine learning methods, are the key contributors to the recent progress in ab initio protein structure prediction, as demonstrated in the recent CASP experiments. Continuing the development of new methods to reliably predict contact maps is essential to further improve ab initio structure prediction.

Described herein is DNCON2, an improved protein contact map predictor based on two-level deep convolutional neural networks. It consists of six convolutional neural networks-the first five predict contacts at 6, 7.5, 8, 8.5 and 10 Å distance thresholds, and the last one uses these five predictions as additional features to predict final contact maps. On the free-modeling datasets in CASP10, 11 and 12 experiments, DNCON2 achieves mean precisions of 35, 50 and 53.4%, respectively, higher than 30.6% by MetaPSICOV on CASP10 dataset, 34% by MetaPSICOV on CASP11 dataset and 46.3% by Raptor-X on CASP12 dataset, when top L/5 long-range contacts are evaluated. We attribute the improved performance of DNCON2 to the inclusion of short- and medium-range contacts into training, two-level approach to prediction, use of the state-of-the-art optimization and activation functions, and a novel deep learning architecture that allows each filter in a convolutional layer to access all the input features of a protein of arbitrary length.

Datasets

The original DNCON dataset consisting of 1426 proteins having length between 30 and 300 residues curated before the CASP10 experiment is used to train and test DNCON2. The protein structures in the dataset were obtained from the Protein Data Bank (PDB) and of 0-2 Å resolution, which were filtered by 30% sequence identity to remove redundancy. 1230 proteins from the dataset are used for training and 196 as the validation set, and the two sets have less than 25 percent sequence identity between them. In addition to the validation dataset, the method is benchmarked using (i) 37 free-modeling domains in the CASP12 experiment, (ii) 30 free-modeling domains in the CASP11 experiment (Kinch et al., 2016) and (iii) 15 free-modeling domains in the CASP10 experiment (Monastyrskyy et al., 2014). These CASP free-modeling datasets have zero or very little identity with the training dataset.

Herein, a pair of residues in a protein is defined to be in contact if their carbon beta atoms (carbon alpha for glycine), are closer than 8 Å in the native structure. Contacts are considered as long-range when the paring residues are separated by at least 24 residues in the protein sequence. Similarly, medium-range contacts are pairs which have sequence separation between 12 and 23 residues and short-range contacts are pairs with sequence separation between 6 and 11 residues. These definitions are consistent with the common standards used in the field (Monastyrskyy et al., 2016).

As a primary evaluation metric of contact prediction accuracy, the precision of top L/5 or L/2 predicted long-range contacts, where L is the length of the protein sequence, are used. The metric has also been the main measure in the recent CASP evaluations (Monastyrskyy et al., 2011, 2014, 2016) and some recent studies (Adhikari et al., 2016). When evaluating the predictions for the proteins in the CASP datasets, we evaluate them at the domain level to be consistent with the past CASP assessments, although all predictions were made on the full target sequences without any knowledge of domains. We used the ConEVA tool to carry out our evaluations (Adhikari et al., 2016).

Input Features

In addition to the existing features used in the original DNCON, new features derived from multiple sequence alignments, coevolution-based predictions and three-state secondary structure predictions from PSIPRED (Jones, 1999) are used. The original DNCON feature set includes length of the protein, secondary structure and solvent accessibility predicted using the SCRATCH suite (Cheng et al., 2005), position specific scoring matrix (PSSM) based features (e.g. PSSM sums and PSSM sum cosines), Atchley factors, and several pre-computed statistical potentials. During experiments, PSSM and amino acid composition are found from the original DNCON feature set were not very useful and hence removed them from the feature list. Besides the DNCON features, the new features include coevolutionary contact probabilities/scores predicted using CCMpred (Seemayer et al., 2014), FreeContact (KajSn et al., 2014), PSICOV (Jones et al., 2012) and alignment statistics such as number of effective sequences, Shannon entropy sum, mean contact potential, normalized mutual information and mutual information generated using the alignment statistics tool ‘alnstat’ (Jones et al., 2014). During experiments, often, PSICOV did not converge when there are too many or too few alignments, especially if the target sequence is long. To guarantee to get some results, a time limit of 24 h is set, and run PSICOV with three convergence parameters (‘d=0.03’, ‘r=0.001’ and ‘r=0.01’) in parallel. If the first prediction (with option d=0.03) finishes within 24 h, we use the prediction, and if not, the second prediction is used and so on. Using all these features above as input, contact maps are predicted at 6, 7.5, 8, 8.5 and 10 Å distance thresholds at first, and then use these five contact-map predictions as additional features to make a second round of prediction. Contact predictions at lower distance thresholds are relatively sparse and include only the residue pairs that are very close in the structure, whereas, contact predictions at higher distance thresholds are denser and provide more positive cases for the deep convolution neural network to learn.

Generating Multiple Sequence Alignments

Generating a diverse/informative multiple sequence alignment with a sufficient number of sequences is critical for generating quality coevolution-based features for contact prediction. On one hand, having too few sequences in the alignment, even though they may be highly diverse, can lead to low contact prediction accuracy. On the other hand, having too many sequences can slow down the process of co-evolution feature generation, creating a bottleneck for an overall structure prediction pipeline. To reliably generate multiple sequence alignments, an alignment method should produce at least some sequences in alignment whenever possible, and does not generate too many more sequences than necessary. Following a similar procedure in (Ovchinnikov et al., 2016) and (Kosciolek and Jones, 2015), HHblits (Remmert et al., 2011) with 60% coverage thresholds is first run, and if a certain number of alignments are not found (usually around 2 L), then JackHMMER (Johnson et al., 2010) with e-value thresholds of 1E⁻²⁰, 1E⁻¹⁰, 1E⁻⁴ and 1 is run until some alignments are found. JackHMMER is not run if HHblits can find at least 5000 sequences. These alignments are used by the three coevolution-based methods (CCMpred, FreeContact and PSICOV) to predict contact probabilities/scores, which are used as two-dimensional features and to generate alignment statistics related features for deep convolutional neural networks.

Deep Convolutional Neural Network Architecture

Convolutional neural networks (CNNs) are widely applied to recognize images with each input image translated into an input volume such that the size of the image are length and width of the volume, and the three channels (hue, saturation and value) represent the depth. Based on such ideas, to build an input volume for each protein of any length, we translate all scalar and one-dimensional input features into two-dimensional features (channels) so that all features (including the ones already in 2D) are in two-dimension and can be viewed as separate channels. While scalar features like sequence length are duplicated to form a two-dimensional matrix (one channel), each one-dimensional feature like solvent accessibility prediction is duplicated across the row and across the column to generate two channels. The size of the channels for a protein is decided by the length of the protein. By having all features in separate input channels in the input volume, each filter in a convolutional layer convolving through the input volume, has access to all the input features, and can learn the relationships across the channels. Compared to the input volumes of images that have three channels, our input volumes have 56 channels.

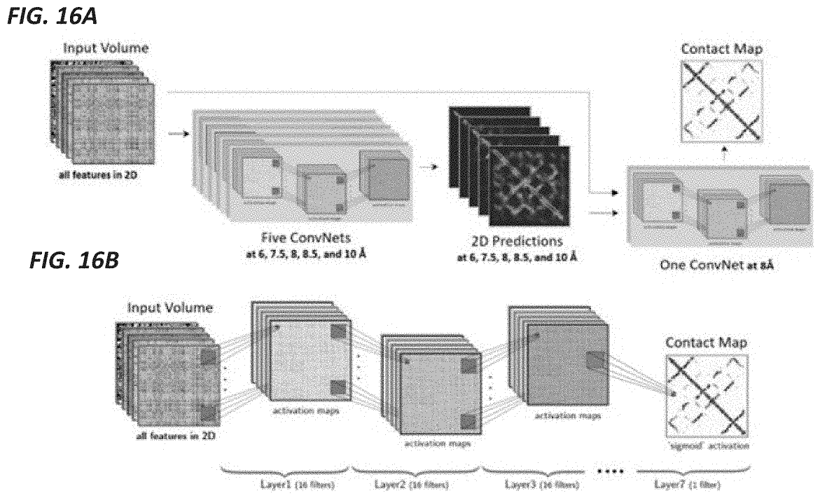

FIGS. 16A-16B illustrate the block diagram of DNCON2's overall architecture. The 2D volumes representing a protein's features are used by five convolution neural networks to predict preliminary contact probabilities at 6, 7.5, 8, 8.5 and 10 Å thresholds at the first level. The preliminary 2D predictions and the input volume are used by a convolutional neural network to predict final contact probability map at the second level. The structure of one deep convolutional neural network in DNCON2 consisting of six hidden convolutional layers with 16 5×5 filters and an output layer consisting of one 5×5 filter to predict a contact probability map.

A total of six CNNs are used, i.e. five in the first level to predict preliminary contact probabilities at 6, 7.5, 8, 8.5 and 10 Å distance thresholds separately by using an input volume of a protein as input, and one in the second level that take both the input volume and the 2D contact probabilities predicted in the first level to make final predictions (FIG. 16A). Each of the six CNN networks have the same architecture, which has six hidden convolutional layers and one output layer consisting of 16 filters of 5 by 5 size and one output layer (FIG. 16B). In the hidden layers, the batch normalization is applied, and ‘Rectified Linear Unit’ (Nair and Hinton, 2010) is used as the activation function. The last output layer consists of one 5 by 5 filter with ‘sigmoid’ as the activation function to predict final contact probabilities. Hence, our deep network can accept a protein of any length and predict a contact map of the same size. We use the Nesterov Adam (nadam) method (Sutskever et al., 2013) as the optimization function to train the network.

Each CNN is trained for a total of 1600 epochs with each epoch of training taking around 2 min. After training, we rank and select best model using the mean precision of top L/5 long-range contacts calculated on the validation dataset of 196 proteins. The raw feature files for all 1426 training proteins use 8 GigaBytes (GB) of disk space and are expanded to around 35 GB when all features are translated to 2D format. To minimize disk input/output, the scalar features and 1D features are translated into 2D, at runtime, in CPU memory. The Keras library (http://keras.io/) along with Tensorflow (www.tensorflow.org) is used to implement the deep CNN networks. The training was conducted on Tesla K20 Nvidia GPUs each having 5 GB of GPU memory, on which, training one model took around 12 h. Finally, an ensemble of 20 trained deep models is used to make final predictions for testing on the CASP datasets.

Results and discussion of the evaluation of DNCON2 is described in Appendix J of U.S. provisional patent application No. 63/280,037, filed on Nov. 16, 2021, and titled “Deep Learning Systems and Methods for Predicting Structural Aspects of Protein-Related Complexes,” the disclosure of which is expressly incorporated herein by reference in its entirety.

### Example 4

Datasets

The current opinion in the bioinformatics community is that protein sequence features still carry important higher-order information concerning residue-residue interactions (Liu et al. (2020)). In particular, the residue-residue coevolution and residue conservation information obtained through multiple sequence alignments (MSAs) has been shown to contain powerful information concerning intra-chain and even inter-chain residue-residue interactions as they yield a compact representation of residues' coevolutionary relationships (Jumper et al. (2021)).

Keeping this in mind, for the training and validation datasets, DIPS-Plus (Morehead et al. (2021)) is chosen, to knowledge the largest feature-rich dataset of protein complexes for protein interface contact prediction to date. In total, DIPS-Plus contains 42,112 binary protein complexes with positive labels (i.e., 1) for each inter-chain residue pair that are found within 6°A of each other in the complex's bound (i.e., structurally-conformed) state. The dataset contains a variety of rich residue level features: (1) an 8-state one-hot encoding of the secondary structure in which the residue is found; (2) a scalar solvent accessibility; (3) a scalar residue depth; (4) a 1×6 vector detailing each residue's protrusion concerning its side chain; (5) a 1×42 vector describing the composition of amino acids towards and away from each residue's side chain; (6) each residue's coordinate number conveying how many residues to which the residue meets a significance threshold, (7) a 1×27 vector giving residues' emission and transition probabilities derived from HH-suite3 (Steinegger et al. (2019)) profile hidden Markov models constructed using MSAs; and (8) amide plane normal vectors for downstream calculation of the angle between each intra-chain residue pair's amide planes.

To compare the performance of DeepInteract with that of state-of-the-art methods, 32 homodimers and heterodimers are selected from the test partition of DIPS-Plus to assess each method's competency in predicting interface contacts. Each method is also evaluated on 14 homodimers and 5 heterodimers with PDB structures publicly available from the 13th and 14th sessions of CASP-CAPRI (Lensink et al. (2019), Lensink et al. (2021)) as these targets are considered by the bioinformatic community to be challenging for existing interface predictors. For any CASP-CAPRI test complexes derived from multimers (i.e., protein complexes that can contain more than two chains), to represent the complex we chose the pair of chains with the largest number of interface contacts. Finally, we use the traditional 55 test complexes from the DB5 dataset (Fout et al. (2017); Townshend et al. (2019); Liu et al. (2020)) to benchmark each heteromer-compatible method.

To expedite training and validation and to constrain memory usage, beginning with all remaining complexes not chosen for testing, all complexes where either chain contains fewer than 20 residues and where the number of possible interface contacts is more than 2562 are filtered out, leaving an intermediate total of 26,504 complexes for training and validation. In DIPS-Plus, binary protein complexes are grouped into shared directories according to whether they are derived from the same parent complex. As such, using a per-directory strategy, 80% of these complexes are randomly designated for training and 20% for validation to restrict overlap between cross-validation datasets. After choosing these targets for testing, we then filter out complexes from training and validation partitions of DIPS-Plus that contain any chain with over 30% sequence identity to any chain in any complex in our test datasets. This threshold of 30% sequence identity is commonly used in the bioinformatics literature (Jordan et al. (2012), Yang et al. (2013)) to prevent large evolutionary overlap between a dataset's cross-validation partitions. However, most existing works for interface contact prediction do not employ such filtering criteria, so the results reported in these works may be over-optimistic by nature. In performing such sequence-based filtering, 15,618 and 3,548 binary complexes are left for training and validation, respectively.

Problem Formulation

Summarized in FIG. 17, DeepInteract, the proposed pipeline for interface contact prediction, has been designed to frame the problem of predicting interface contacts in silico as a two-part task: The first part is to use attentive graph representation learning to inductively learn new node-level representations [symbol] ∈ [symbol] and [symbol] ∈ [symbol] for a pair of graphs representing two protein chains. The second part is to channel-wise interleave [symbol] and [symbol] into an interaction tensor [symbol] ∈ [symbol] , where [symbol] ∈ [symbol] and [symbol] ∈ [symbol] are the numbers of amino acid residues in the first and second input protein chains, respectively, and [symbol] ∈ [symbol] is the number of hidden channels in both [symbol] and [symbol] . Interaction tensors such as [symbol] are used as input to the interaction module, a convolution-based dense predictor of inter-graph node-node interactions. Each protein chain in an input complex is denoted as a graph [symbol] with edges [symbol] between the k-nearest neighbors of its nodes [symbol] , with nodes corresponding to the chain's amino acid residues represented by their Ca atoms. In this setting, let k=20 as favorable cross entropy loss is observed on the validation dataset with this level of connectivity. It is noted that this level of graph connectivity has also proven to be advantageous for prior works developing deep learning approaches for graph-based protein representations (Fout et al. (2017); Ingraham et al. (2019)).

Geometric Transformer Architecture

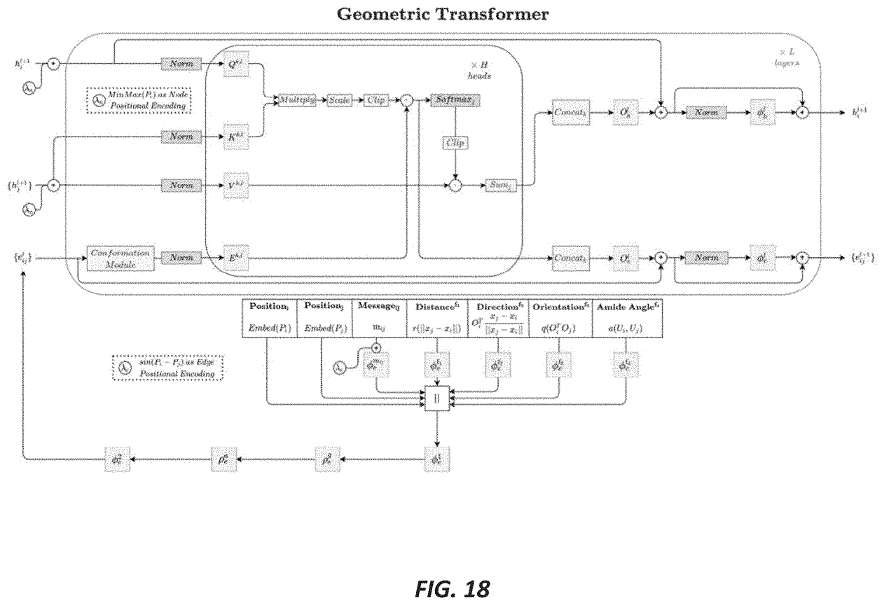

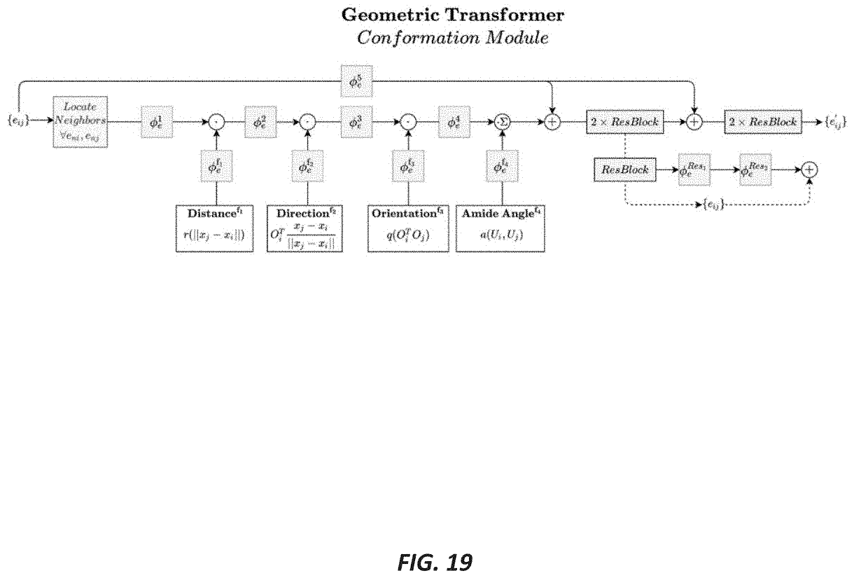

Hypothesizing that a self-attention mechanism that evolves proteins' physical geometries is a key component missing from existing interface contact predictors, the Geometric Transformer, a graph neural network explicitly designed for capturing and iteratively evolving protein geometric features, is designed. As shown in FIG. 18, the Geometric Transformer expands upon the existing Graph Transformer architecture (Dwivedi & Bresson (2021)) by introducing (1) an edge initialization module, (2) an edge-wise positional encoding (EPE), and (3) a geometry-evolving conformation module employing repeated geometric feature gating (GFG). Moreover, the Geometric Transformer includes subtle architectural enhancements to the original Transformer architecture (Vaswani et al. (2017)) such as moving the network's first normalization layer to precede any affinity score computations for improved training stability (Hussain et al. (2021)). The Geometric Transformer is the first deep learning model that applies multi-head attention to the task of partner-specific protein interface contact prediction. The following sections serve to distinguish the Geometric Transformer of this example from other Transformer-like architectures by describing its new neural network modules for geometric self-attention.

Edge Initialization Module

To accelerate its training, the Geometric Transformer first embeds each edge e∈ [symbol] with the initial edge representation

cᵢⱼ=ϕₑ¹([p₁∥p₂∥ϕₑ^{m}^{ᵢⱼ}(mᵢⱼ∥λₑ)∥ϕₑ^{f}^{₁}(f₁)∥ϕₑ^{f}^{₂}(f₂)∥ϕₑ^{f}^{₃}(f₃)∥ϕₑ^{f}^{₄}(f₄)]) (1)

eᵢⱼ=ϕₑ²(pₑ^{a}(pₑ^{g}(cᵢⱼ))) (2)

where ϕₑ^{b}, refers to the i′th edge information update function such as a multi-layer perceptron; denotes channel-wise concatenation; p₁ and p₂, respectively, are trainable one-hot vectors indexed by Pᵢ and Pⱼ, the positions of nodes i and nodes j in the chain's underlying amino acid sequence; my are any user-predefined features for e (in this case the normalized Euclidean distances between nodes i and nodes j); λₑ are edge-wise sinusoidal positional encodings sin(Pᵢ−Pⱼ) for e; f1, f2, f3, and f4, in order, are the four protein-specific geometric features; pₑ^{a} and pₑ^{g} and are feature addition and channel-wise gating functions, respectively.

Conformation Module

The role of the Geometric Transformer's subsequent conformation module, as illustrated in FIG. 4, is for it to learn how to iteratively evolve geometric representations of protein graphs by applying repeated gating to our initial edge geometric features f1, f2, f3, and f4. To do so, the conformation module updates eᵢⱼ by introducing the notion of a geometric neighborhood of edge e, treating e as a pseudo-node. Precisely, [symbol] , the edge geometric neighborhood of e, is defined as the 2n edges

[symbol]

ₖ={eₙ_{₁}ᵢ,eₙ_{₂}ⱼ|(n₁,n₂∈ [symbol] ₖ) and (n₁,n₂≠i,j)}, (3)

where [symbol] ₖ⊂ [symbol] are the source nodes for incoming edges on edge e's source and destination nodes. The intuition behind updating each edge according to its 2n nearest neighboring edges is that the geometric relationship between a residue pair, described by their mutual edge's features, can be influenced by the physical constraints imposed by proximal residue-residue geometries. As such, these nearby edges are used during geometric feature updates. In the conformation module, the iterative processing of all geometric neighborhood features for edge e can be represented as

$${O}_{ij} = {\sum }_{k ∈ {𝔼}_{k}} [ ( {ϕ}_{e}^{n} ( {e}_{ij , k}^{n} ) ⊙ {ϕ}_{e}^{{f}_{n}} ( {f}_{n} ) ) , ∀ n ∈ 𝔽 ] \qquad ( 4 )$$

$${e}_{ij} = 2 \times {ResBlock}_{2} ( {ϕ}_{e}^{5} ( {e}_{ij} ) + 2 \times {ResBlock}_{1} ( {ϕ}_{e}^{5} ( {e}_{ij} ) + {O}_{ij} ) ) , \qquad ( 5 )$$

where [symbol] are the indices of the geometric features {f1; f2; f3; f4}; ⊙ is element-wise multiplication; e_{ij,k}ⁿ is neighboring edge eₖ's representation gaiting with fn-1; and 2×ResBlock, represents the l′th application of two unique, successive residual blocks, each defined as ResBlock(x)=ϕₑ^{Res}^{₂}(ϕₑ^{Res}^{₁}(x))+x. By way of their construction, each of the selected edge geometric features is translation and rotation invariant to the network's input space. These features are coupled with the choice of node-wise positional encodings to attain canonical invariant local frames for each residue to encode the relative poses of features in our protein graphs. In doing so, many of the benefits of employing equivariant representations are employed while reducing the large memory requirements they typically induce, to yield a robust invariant representation of each input protein.

Remaining Transformer Initializations and Operations

For the initial node features used within the Geometric Transformer, each of DIPS-Plus' residue-level features is included. Additionally, initial min-max normalizations of each residue's index in Pᵢ are appended to each node as node-wise positional encodings. For the remainder of the Geometric Transformer's operations, the network's order of operations closely follows the definitions given by Dwivedi & Bresson (2021) for the Graph Transformer, with an exception being that the first normalization layer now precedes any affinity score calculations.

Interaction Module

Upon applying multiple layers of the Geometric Transformer to each pair of input protein chains, the Geometric Transformer's learned node representation [symbol] and [symbol] are channel-wise interleaved into [symbol] to serve as input to the interaction module, consisting of a dilated ResNet module adapted from Chen et al. (2021). The core residual network component in this interaction module consists of four residual blocks differing in the number of internal layers. Each residual block is comprised of several consecutive instance normalization layers and convolutional layers with 64 kernels of size 3×3. The number of layers in each block represents the number of 2D convolution layers in the corresponding component. The final values of the last convolutional layer are added to the output of a shortcut block, which is a convolutional layer with 64 kernels of size 1×1. A squeeze-andexcitation (SE) block (Hu et al. (2018)) is added at the end of each residual block to adaptively recalibrate its channel-wise feature responses. Ultimately, the output of the interaction module is a probability-valued [symbol] matrix that can be viewed as an inter-chain residue binding heatmap.

Results and discussion of the evaluation of DeepInteract is described in Appendix G of U.S. provisional patent application No. 63/280,037, filed on Nov. 16, 2021, and titled “Deep Learning Systems and Methods for Predicting Structural Aspects of Protein-Related Complexes,” the disclosure of which is expressly incorporated herein by reference in its entirety.

### Example 5

Predicting the quaternary structure of protein complex is an important problem. Inter-chain residue-residue contact prediction can provide useful information to guide the ab initio reconstruction of quaternary structures. However, few methods have been developed to build quaternary structures from predicted inter-chain contacts. Here, we develop the first method based on gradient descent optimization (GD) to build quaternary structures of protein dimers utilizing inter-chain contacts as distance restraints. We evaluate GD on several datasets of homodimers and heterodimers using true/predicted contacts and monomer structures as input. GD consistently performs better than both simulated annealing and Markov Chain Monte Carlo simulation. Starting from an arbitrarily quaternary structure randomly initialized from the tertiary structures of protein chains and using true inter-chain contacts as input, GD can reconstruct high-quality structural models for homodimers and heterodimers with average TM-score ranging from 0.92 to 0.99 and average interface root mean square distance from 0.72 Å to 1.64 Å. On a dataset of 115 homodimers, using predicted inter-chain contacts as restraints, the average TM-score of the structural models built by GD is 0.76. For 46% of the homodimers, high-quality structural models with TM-score ≥0.9 are reconstructed from predicted contacts. There is a strong correlation between the quality of the reconstructed models and the precision and recall of predicted contacts. Only a moderate precision or recall of inter-chain contact prediction is needed to build good structural models for most homodimers. Moreover, GD improves the quality of quaternary structures predicted by AlphaFold2 on a Critical Assessment of Techniques for Protein Structure Prediction-Critical Assessments of Predictions of Interactions dataset.

Determination of interactions between protein chains in a protein complex is important for understanding protein function and cellular processes and can play significant roles in designing and discovering new drugs. Detailed protein-protein interactions are represented by the three-dimensional shape of a complex consisting of interacting proteins (i.e., quaternary structure). Experimental techniques such as X-ray crystallography and nuclear magnetic resonance (NMR) can determine the quaternary structure of protein complexes with high accuracy. However, these experimental approaches are costly and time-consuming, and therefore cannot be applied to most protein complexes. Therefore, computational modeling approaches, which provide a faster and inexpensive way to predict quaternary structures, have become increasingly popular and important.

Described herein is an ab initio gradient descent optimization-based method (GD) to construct quaternary structures of protein dimers from inter-chain contacts. The proposed method is first tested to determine if it can generate high quality structures of protein dimers using true contacts. Then, it is applied to construct quaternary structures of homodimers from predicted, noisy, and incomplete contacts. To rigorously benchmark its performance, a Markov Chain simulation method (MC) based on RosettaDock is implemented and a simulated annealing method based on Crystallography and NMR System (CNS) is applied to reconstruct protein complex structures from inter-protein contacts and compare them with GD. The three methods are evaluated on several in-house datasets consisting of 233 homodimers and heterodimers in total as well as on a standard dataset of 32 heterodimers with true or predicted contacts. GD consistently performs better than MC and CNS on all the datasets. It can reconstruct high-quality structures from true inter-chain contacts and good structures for most homodimers when predicted contacts are only moderately accurate. Finally, GD is applied to 28 homodimers used in the several recent CASP-CAPRI experiments and seven homomeric targets of the latest 2020 CASP14-CAPRI experiment to investigate how the quality of input (i.e., tertiary structure of monomers predicted by AlphaFold2 and inter-chain contact prediction) influences its performance.

Inter-Chain Contacts Dimer Datasets

Two residues from two protein chains in a dimer are considered an inter-chain contact if any two heavy atoms from the two residues have a distance less than or equal to 6 Å. True contacts of a dimer with the known quaternary structure in the PDB are identified according to the coordinates of atoms in the PDB file of the dimer.

Several in-house datasets of protein homodimers and heterodimers with true and/or predicted inter-protein contacts are used as well as a standard datasets consisting of 32 heterodimers (Std32) to evaluate the methods. The first in-house dataset has 44 homodimers randomly selected from the Homo_Std curated from the 3D Complex database, each of which have 39-621 true contacts (called Homo44). The second in-house data includes 115 homodimers (called Homo115) selected from Homo_Std, each of which has at least 21 predicted inter-chain contacts with a probability of 20.5. An in-house deep learning method—ResCon is applied to predict inter-chain contacts for the dimers in Homo115. Homo115 is divided into three subsets (Set A, Set B, and Set C) according to the size of interfaces. Set A has 40 protein complexes with small interaction interfaces consisting of 14-68 true inter-chain contacts. Set B consists of 37 complexes having medium interaction interfaces with 69-129 true contacts. Set C consists of 38 complexes having large interaction interfaces with 131-280 true contacts. The third in-house dataset contains 73 heterodimers (called Hetero73) curated from the PDB, in which the sum of the lengths of the two chains is less than or equal to 400. The heterodimers in Hetero73 have 2 to 255 true inter-protein contacts.

Moreover, GD is also tested on 28 homodimers from the recent CASP-CAPRI joint experiment used in DeepHomo (called CASP-CAPRI dataset), and seven homodimers from the latest CASP14-CAPRI experiment collected from five homodimeric targets (T1054, T1078, T1032, T1083, T1087) and two homotrimers (T1050, and T1070; called CASP14-CAPRI dataset).

Gradient Descent Cost Function and Optimization

The inter-chain contacts are used as distance restraints for the gradient descent method to build the structures of protein dimers. The cost function to measure the satisfaction of the distance between any two residues in contact to guide the structural modeling is defined as follows:

$$f ( x ) = \{ \begin{matrix}{( \frac{x - l b}{sd} )}^{2} & x < l b \\ 0 & l b \leq x \leq ub \\ {( \frac{x - ub}{sd} )}^{2} & ub < x < ub + sd \\ \frac{1}{sd} ( x - ( ub + sd ) ) & x > ub + sd\end{matrix}$$

Here, lb and ub represent the lower bound and upper bound of the distance (x) between two residues that are assumed to be in contact. As mentioned earlier, two residues are considered in contact if the distance between their heavy atoms is less than 6 Å. However, to simplify the process of restraint preparation, two residues are considered in contact if the distance between their C_{β} atoms (C_{α} for glycine) is less than 6 Å. The lower bound (lb) is empirically set to 0 and the upper bound (ub) to 6 Å. sd is the standard deviation, which is set to 0.1. Based on this cost function, if the distance between two residues in contact is ≤6 Å, that is, the contact restraint is satisfied, and the cost is 0.

The complete contact cost function for a structural model of a dimer to be minimized is the sum of the costs for all contacts used in modeling (called contact energy). For simplicity, all restraints have equal weights and play equally important roles in modeling. The contact energy function is differentiable with respect to the distances between residues and coordinates of atoms of the residues, and therefore it can be minimized by a gradient descent iterative algorithm (GD), that is, Limited-memory Broyden-Fletcher-Goldfarb-Shanno algorithm (L-BFGS)) used in this study.

GD is implemented on top of pyRosetta. The total energy function for the structural optimization is the combination of the contact energy and the talaris2013 potentials, which works better than the contact energy alone in our experiments. The input to the algorithm includes inter-chain contacts and an initial random conformation of a dimer initialized from the tertiary structures of individual protein chains (monomers) of the dimer. The tertiary structure of monomer can be either in the bound state or unbound state. It can be either an experimentally determined true structure or a predicted structure. A predicted structure is considered in the unbound state because the existing methods generally predicts the tertiary structure of a chain without considering its partner. An initial conformation of a protein dimer is generated by making 40 random, rigid rotations and translations ranging from 1°-360° and 1 Å-20 Å of the tertiary structures of the two chains in a dimer after putting them in the same coordinate system. Specifically, the tertiary structure of each protein chain is rotated and translated arbitrarily along the line connecting the centers of the two chains, aiming to make the two protein chains facing each other.

Then, 6000 iterations of the gradient descent optimization (i.e., L-BFGS) are carried out to generate new structural models. In most cases tested, it converged after only 1000 iterations. Since the quality of the final structure is influenced by the initial structure, the optimization process is carried out 20 times, each with a random structure as the start point. The optimized structure with the lowest energy is selected as the final predicted structure of a dimer. For 20 runs, the total number of iterations of the gradient descent optimization is 1.2×10⁵.

Markov Chain Monte Carlo Optimization

A Rosetta protocol in pyRosetta based on Metropolis-Hasting sampling is applied to implement a Markov chain Monte Carlo (MC) optimization to reconstruct complex structures according to the Boltzmann distribution. An initial conformation of a dimer is generated in the same way as in the GD algorithm. Starting from the initial conformation, a low-resolution rigid-body search is employed to rotate and translate one chain around the surface of the other chain to generate new structures in the MC optimization. Five hundred Monte Carlo moves are attempted. Each move is accepted or rejected based on the standard Metropolis acceptance criterion.

After the low-resolution search, back-bone and side-chain conformations are further optimized with the Newton minimization method in a high-resolution refinement process, in which the gradient of the scoring function dictates the direction of the starting point in the rigid-body translation/rotation space. This minimization process is repeated 50 times to detect the local minimum of the energy function that may have similar performance as the global minimum.

The MC method above is implemented using high-resolution and low-resolution docking protocols in RosettaDock to optimize the same energy function used in the GD method. Low-resolution docking is performed using the DockingLowRes protocol, whereas DockMCMProtocol is used to perform high-resolution docking. For a dimer, 10⁵ to 10⁷ rounds of MC optimization with different initial conformations are executed to generate structural models. At the end, 10⁵ to 10⁷ models are generated, among which the model with the lowest energy is selected as the final prediction.

Simulated Annealing Optimization Based on Crystallography and NMR System

This structure optimization method, Con_Complex in the DeepComplex package, is implemented on top of the CNS that uses a simulated annealing protocol to search for quaternary structures that satisfy inter-chain contacts. This method takes the PDB files of monomers (protein chains) in a protein multimer (e.g., homodimer) and the true or predicted inter-protein contacts as input to reconstruct the structure of the multimer without altering the shape of the structure of the monomer. The inter-protein contacts are converted into distance restraints used by CNS. This process generates 100 structural models and then picks five models with lowest CNS energy. It is worth noting that this method can handle the reconstruction of the quaternary structure of any multimer consisting of multiple identical or different chains. Because inter-chain contacts are the main restraints to guide structure modeling, the performance of this method mostly depends on the quality of the inter-protein contact predictions.

### CONCLUSION

As described herein, the first gradient descent distance optimization (GD)-based method to reconstruct quaternary structure of protein dimers from inter-protein contacts is designed and developed. It is compared with the Markov Chain Monte Carlo and simulated annealing optimization methods adapted to address the problem. GD performs consistently better than the other two methods in reconstructing quaternary structures of dimers from either true or predicted inter-chain contacts. GD can reconstruct high-quality structures for almost all homodimers and heterodimers from true inter-chain contacts and can build good structural models for many homodimers from only predicted inter-chain contacts, demonstrating distance-based optimizations are useful tools for predicting the quaternary structures. Moreover, it is shown that the contact density, size of interaction interface, precision and recall of predicted contacts, and threshold of selecting contacts as restraints influence the accuracy of reconstructed models. Particularly, when the precision and recall of predicted contacts reach a moderate level (e.g., >20%), GD can construct good models for most homodimers, demonstrating that predicting inter-chain contacts or even distances and distance-based optimization are a promising ab initio approach to predicting the quaternary structures of protein complexes.

Results and discussion of the evaluation of the GD to construct quaternary structures of protein dimers is described in Appendix H of U.S. provisional patent application No. 63/280,037, filed on Nov. 16, 2021, and titled “Deep Learning Systems and Methods for Predicting Structural Aspects of Protein-Related Complexes,” the disclosure of which is expressly incorporated herein by reference in its entirety.

### REFERENCES

[1] Utsumi, S. & Matsumura, Y. Structure-Function Relationships. Food proteins and their applications 80, 257 (1997).
[2] Spirin, V. & Mirny, L A. Protein complexes and functional modules in molecular networks. Proceedings of the national Academy of sciences 100, 12123-12128 (2003).
[3] Eickholt, J. & Cheng, J. Predicting protein residue-residue contacts using deep networks and boosting. Bioinformatics 28, 3066-3072 (2012).
[4] Adhikari, B., Hou, J. & Cheng, J. DNCON2: improved protein contact prediction using two-level deep convolutional neural networks. Bioinformatics 34, 1466-1472 (2018).
[5] Wang, S., Sun, S., Li, Z., Zhang, R. & Xu, J. Accurate de novo prediction of protein contact map by ultra-deep learning model. PLoS computational biology 13, e1005324 (2017).
[6] Li, Y., Hu, J., Zhang, C., Yu, D.-J. & Zhang, Y. ResPRE: high-accuracy protein contact prediction by coupling precision matrix with deep residual neural networks. Bioinformatics 35, 4647-4655 (2019).
[7] Wu, T., Guo, Z., Hou, J. & Cheng, J. DeepDist: real-value inter-residue distance prediction with deep residual network. bioRxiv (2020).
[8] Senior, A. W. et al. Improved protein structure prediction using potentials from deep learning. Nature, 1-5 (2020).
[9] Adhikari, B. & Cheng, J. CONFOLD2: improved contact-driven ab initio protein structure modeling. BMC bioinformatics 19, 22 (2018).
[10] Rohl, C. A., Strauss, C. E., Misura, K. M. & Baker, D. in Methods in enzymology Vol. 383 66-93 (Elsevier, 2004).
[11] Kandathil, S. M., Greener, J. G., Lau, A. M. & Jones, D. T. Ultrafast end-to-end protein structure prediction enables high-throughput exploration of uncharacterized proteins. Proceedings of the National Academy of Sciences 119 (2022).
[12] Yang, J. et al. Improved protein structure prediction using predicted interresidue orientations. Proceedings of the National Academy of Sciences 117, 1496-1503 (2020).
[13] Xu, J. & Wang, S. Analysis of distance-based protein structure prediction by deep learning in CASP13. Proteins: Structure, Function, and Bioinformatics 87, 1069-1081 (2019).
[14] Jumper, J. et al. Highly accurate protein structure prediction with AlphaFold. Nature 596, 583-589 (2021).
[15] Zemla, A. LGA: a method for finding 3D similarities in protein structures. Nucleic acids research 31, 3370-3374 (2003).
[16] Evans, R. et al. Protein complex prediction with AlphaFold-Multimer. BioRxiv (2021).
[17] Gao, M., Nakajima An, D., Parks, J. M. & Skolnick, J. AF2Complex predicts direct physical interactions in multimeric proteins with deep learning. Nature Communications 13, 1-13 (2022).
[18] Zeng, H. et al. ComplexContact: a web server for inter-protein contact prediction using deep learning. Nucleic acids research 46, W432-W437 (2018).
[19] Yan, Y. & Huang, S.-Y. Accurate prediction of inter-protein residue-residue contacts for homo-oligomeric protein complexes. Briefings in bioinformatics 22, bbab038 (2021).
[20] Roy, R. S., Quadir, F., Soltanikazemi, E. & Cheng, J. A deep dilated convolutional residual network for predicting interchain contacts of protein homodimers. bioRxiv (2021).
[21] Xie, Z. & Xu, J. Deep graph learning of inter-protein contacts. Bioinformatics 38, 947-953 (2022).
[22] Rao, R. M. et al. in International Conference on Machine Learning. 8844-8856 (PMLR).
[23] He, K., Zhang, X., Ren, S. & Sun, J. in Proceedings of the IEEE conference on computer vision and pattern recognition. 770-778.
[24] Quadir, F., Roy, R. S., Halfmann, R. & Cheng, J. DNCON2_Inter: predicting interchain contacts for homodimeric and homomultimeric protein complexes using multiple sequence alignments of monomers and deep learning. Scientific reports 11, 1-10 (2021).
[25] Quadir, F., Roy, R. S., Soltanikazemi, E. & Cheng, J. DeepComplex: a web server of predicting protein complex structures by deep learning inter-chain contact prediction and distance-based modelling. Frontiers in Molecular Biosciences, 827 (2021).
[26] Jones, D. T., Singh, T., Kosciolek, T. & Tetchner, S. MetaPSICOV: combining coevolution methods for accurate prediction of contacts and long range hydrogen bonding in proteins. Bioinformatics 31, 999-1006 (2015).
[27] Guo, Z., Wu, T., Liu, J., Hou, J. & Cheng, J. Improving deep learning-based protein distance prediction in CASP14. Bioinformatics 37, 3190-3196 (2021).
[28] Hunter, J. D. Matplotlib: A 2D graphics environment. Computing in science & engineering 9, 90-95 (2007).
[29] Zhang, Y. & Skolnick, J. Scoring function for automated assessment of protein structure template quality. Proteins: Structure, Function, and Bioinformatics 57, 702-710 (2004).
[30] Goodfellow, I. J., Warde-Farley, D., Mirza, M., Courville, A. & Bengio, Y. Maxout networks. arXiv preprint arXiv:1302.4389 (2013).
[31] Li, Y., Zhang, C., Bell, E. W., Yu, D. J. & Zhang, Y. Ensembling multiple raw coevolutionary features with deep residual neural networks for contact-map prediction in CASP13. Proteins: Structure, Function, and Bioinformatics 87, 1082-1091 (2019).
[32] Mao, W., Ding, W., Xing, Y. & Gong, H. AmoebaContact and GDFold as a pipeline for rapid de novo protein structure prediction. Nature Machine Intelligence, 1-9 (2019).
[33] Ulyanov, D., Vedaldi, A. & Lempitsky, V. Instance normalization: The missing ingredient for fast stylization. arXiv preprint arXiv:1607.08022 (2016).
[34] Hu, J., Shen, L & Sun, G. in Proceedings of the IEEE conference on computer vision and pattern recognition. 7132-7141.
[35] Woo, S., Park, J., Lee, J.-Y. & Kweon, I. S. in Proceedings of the European conference on computer vision (ECCV). 3-19.
[36] Soltanikazemi, E., Quadir, F., Roy, R. S., Guo, Z. & Cheng, J. Distance-based reconstruction of protein quaternary structures from inter-chain contacts. Proteins: Structure, Function, and Bioinformatics 90, 720-731 (2022).
[37] Bhagwat, M. & Aravind, L in Comparative genomics 177-186 (Springer, 2007).
[38] Mirdita, M. et al. Uniclust databases of clustered and deeply annotated protein sequences and alignments. Nucleic acids research 45, D170-D176 (2017).
[39] Bryant, P., Pozzati, G. & Elofsson, A. Improved prediction of protein-protein interactions using AlphaFold2. Nature Communications 13, 1-11 (2022).
[40] Remmert, M., Biegert, A., Hauser, A. & Söding, J. HHblits: lightning-fast iterative protein sequence searching by HMM-HMM alignment. Nature methods 9, 173 (2012).
[41] Steinegger, M., Mirdita, M. & Söding, J. Protein-level assembly increases protein sequence recovery from metagenomic samples manyfold. Nature methods 16, 603-606 (2019).
[42] Green, A. G. et al. Large-scale discovery of protein interactions at residue resolution using co-evolution calculated from genomic sequences. Nature communications 12, 1-12 (2021).
[43] Seemayer, S., Gruber, M. & Söding, J. CCMpred-fast and precise prediction of protein residue-residue contacts from correlated mutations. Bioinformatics 30, 3128-3130 (2014).
[44] Steinegger, M. & Söding, J. MMseqs2 enables sensitive protein sequence searching for the analysis of massive data sets. Nature biotechnology 35, 1026-1028 (2017).
[45] Gao, M. & Skolnick, J. APoc: large-scale identification of similar protein pockets. Bioinformatics 29, 597-604 (2013).
[46] Zhao, Z. & Gong, X. Protein-protein interaction interface residue pair prediction based on deep learning architecture. IEEE/ACM transactions on computational biology and bioinformatics 16, 1753-1759 (2017).
[47] Guo, Z. & Cheng, J. Prediction of inter-chain distance maps of protein complexes with 2D attention-based deep neural networks. BioinfoMachineLeaming/CDPred. doi:10.5281/zenodo.7218709. (2022).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computer-implemented method for predicting inter-chain distances of protein-related complexes comprising:
receiving data associated with a protein-related complex, wherein the data associated with the protein-related complex comprises at least one of a tertiary structural feature and a multiple sequence alignment (MSA)-derived feature;
inputting the data associated with the protein-related complex into a deep learning model; and
predicting, using the deep learning model, an inter-chain distance map for the protein-related complex.

2. The computer-implemented method of claim 1, wherein the tertiary structural feature comprises an intra-chain distance map for at least one monomer of the protein-related complex.

3. The computer-implemented method of claim 1, wherein the MSA-derived feature comprises a plurality of residue-residue co-evolutionary scores for at least one monomer of the protein-related complex.

4. The computer-implemented method of claim 1, wherein the MSA-derived feature comprises a position-specific scoring matrix (PSSM) for at least one monomer of the protein-related complex.

5. The computer-implemented method of claim 1, wherein the data associated with the protein-related complex comprises the tertiary structural feature and the MSA-derived feature.

6. The computer-implemented method of claim 1, wherein the protein-related complex is a homodimer, and wherein the data associated with the protein-related complex comprises information related to a single monomer.

7. The computer-implemented method of claim 1, wherein the protein-related complex is a heterodimer, and wherein the data associated with the protein-related complex comprises respective information related to a plurality of monomers.

8. The computer-implemented method of claim 1, wherein the protein-related complex comprises a first protein and a second protein, the first protein comprising a first chain of residues, and the second protein comprising a second chain of residues.

9. The computer-implemented method of claim 8, wherein the inter-chain distance map comprises a plurality of residue-to-residue distances between residues of the first and second proteins.

10. The computer-implemented method of claim 1, wherein the protein-related complex comprises a protein and a ligand.

11. The computer-implemented method of claim 10, wherein the inter-chain distance map comprises a plurality of atom-to-atom distances between atoms of the protein and the ligand.

12. The computer-implemented method of claim 1, wherein the inter-chain distance map is a heavy atom distance map.

13. The computer-implemented method of claim 1, wherein the inter-chain distance map is a Ce distance map.

14. The computer-implemented method of claim 1, wherein the inter-chain distance map for the protein-related complex is at least one matrix comprising a respective probability of a residue-residue distance between respective residues of a pair of monomers of the protein-related complex in each of a plurality of distance bins.

15. The computer-implemented method of claim 1, wherein the deep learning model comprises an artificial neural network (ANN), the ANN comprising a plurality of layers, the layers comprising at least two of a convolution layer, an attention layer, a dense layer, a dropout layer, a residual layer, a normalization layer, and an embedding layer.

16. The computer-implemented method of claim 15, wherein the ANN comprises a transformer network.

17. The computer-implemented method of claim 16, wherein the transformer network comprises a plurality of 2D transformer blocks, the 2D transformer blocks comprising a plurality of attention generation layers, a plurality of attention convolution layers, and a plurality of projection layers.

18. The computer-implemented method of claim 1, wherein the deep learning model comprises an attention-powered residual neural network.

19. The computer-implemented method of claim 1, wherein the deep learning model comprises a dilated convolutional residual neural network.

20. The computer-implemented method of claim 1, wherein the deep learning model comprises a plurality of convolutional neural networks (CNNs).

21. The computer-implemented method of claim 1, wherein the deep learning model comprises a graph neural network (GNN).

22. The computer-implemented method of claim 1, further comprising generating a three-dimensional (3D) structure for the protein-related complex using the inter-chain distance map as a restraint.

23. The computer-implemented method of claim 22, wherein the protein-related complex comprises a first protein and a second protein, the first protein comprising a first chain of residues, the second protein comprising a second chain of residues, and the inter-chain distance map for the protein-related complex comprising a plurality of residue-to-residue distances between residues of the first and second proteins.

24. The computer-implemented method of claim 23, wherein the 3D structure is a quaternary structure.

25. The computer-implemented method of claim 22, wherein the protein-related complex comprises a protein and a ligand, the inter-chain distance map comprising a plurality of atom-to-atom distances between atoms of the protein and the ligand.

26. The computer-implemented method of claim 22, wherein the 3D structure is generated using a gradient decent (GD) optimization.

27. The computer-implemented method of claim 22, wherein the 3D structure is generated using a simulated annealing algorithm.

28. The computer-implemented method of claim 22, wherein the 3D structure is generated using a Monte Carlo simulation.

29. The computer-implemented method of claim 22, wherein the 3D structure is generated using deep reinforcement learning (DRL).

30. The computer-implemented method of claim 1, further comprising training the deep learning model using a dataset, wherein the dataset comprises known inter-chain parameters for a plurality of known protein-related complexes.

31. The computer-implemented method of claim 30, wherein each known protein-related complex comprises a first protein and a second protein, the first protein comprising a first chain of residues, the second protein comprising a second chain of residues, and wherein the inter-chain parameters comprise a plurality of residue-to-residue distances between residues of the first and second proteins.

32. The computer-implemented method of claim 30, wherein each known protein-related complex comprises a first protein and a second protein, the first protein comprising a first chain of residues, the second protein comprising a second chain of residues, and wherein the inter-chain parameters comprise a plurality of residue-to-residue contacts between residues of the first and second proteins.

33. The computer-implemented method of claim 30, wherein the protein-related complex comprises a protein and a ligand, and wherein the inter-chain parameters comprise a plurality of atom-to-atom distances between atoms of the protein and the ligand.

34. The computer-implemented method of claim 30, wherein the protein-related complex comprises a protein and a ligand, and wherein the inter-chain parameters comprise a plurality of atom-to-atom contacts between atoms of the protein and the ligand.

35. A system for predicting inter-chain distances of protein-related complexes comprising:
a deep learning model; and
a processor and a memory, the memory having computer-executable instructions stored thereon that, when executed by the processor, cause the processor to:
input data associated with a protein-related complex into the deep learning model, wherein the data associated with the protein-related complex comprises at least one of a tertiary structural feature and a multiple sequence alignment (MSA)-derived feature; and
receive, from the deep learning model, an inter-chain distance map for the protein-related complex, wherein the inter-chain distance map for the protein-related complex is predicted by the deep learning model.

36. The system of claim 35, wherein the processor comprises a plurality of processors.

37. The system of claim 36, wherein the processors are part of a distributed computing architecture.

38. The system of claim 35, wherein the memory comprises a plurality of memories.

39. The system of claim 35, wherein the data associated with the protein-related complex comprises the tertiary structural feature and the MSA-derived feature.

40. The system of claim 35, wherein the deep learning model comprises an attention-powered residual neural network.

41. The system of claim 35, wherein the memory has further computer-executable instructions stored thereon that, when executed by the processor, cause the processor to generate a three-dimensional (3D) structure for the protein-related complex using the inter-chain distance map as a restraint.

42. The system of claim 35, wherein the memory has further computer-executable instructions stored thereon that, when executed by the processor, cause the processor to train the deep learning model using a dataset, wherein the dataset comprises known inter-chain parameters for a plurality of known protein-related complexes.

43. A computer-implemented method for predicting a three-dimensional (3D) structure of protein-related complexes comprising:
receiving first data associated with a protein-related complex;
receiving second data comprising a plurality of inter-chain parameters for the protein-related complex;
performing structure optimization on the first data using the second data as a restraint; and
predicting the 3D structure of the protein-related complex based on the structure optimization, wherein the structure optimization comprises gradient descent (GD) optimization.

44. The computer-implemented method of claim 43, wherein the protein-related complex comprises a first protein and a second protein, the first protein comprising a first chain of residues, and the second protein comprising a second chain of residues.

45. The computer-implemented method of claim 44, wherein the second data comprises a plurality of residue-to-residue distances between residues of the first and second proteins.

46. The computer-implemented method of claim 44, wherein the second data comprises a plurality of residue-to-residue contacts between residues of the first and second proteins.

47. The computer-implemented method of claim 43, wherein the protein-related complex comprises a protein and a ligand.

48. The computer-implemented method of claim 47, wherein the second data comprises a plurality of atom-to-atom distances between atoms of the protein and the ligand.

49. The computer-implemented method of claim 47, wherein the second data comprises a plurality of atom-to-atom contacts between atoms of the protein and the ligand.

50. A system for predicting a three-dimensional (3D) structure of protein-related complexes comprising:
a processor and a memory, the memory having computer-executable instructions stored thereon that, when executed by the processor, cause the processor to:
receive first data associated with a protein-related complex;
receive second data comprising a plurality of inter-chain parameters for the protein-related complex;
perform structure optimization on the first data using the second data as a restraint; and
predict the 3D structure of the protein-related complex based on the structure optimization.
